(19) United States
(12) Patent Application Publication
(10) Pub. No.: US 2024/0100069 A1
(43) Pub. Date: Mar. 28, 2024

(54) Methods and Compositions for Treating Amyotrophic Lateral Sclerosis

(71) Applicant: Amylyx Pharmaceuticals, Inc., Cambridge, MA (US)

(72) Inventors: Joshua Cohen, Canton, MA (US); Justin Klee, Cambridge, MA (US)

(21) Appl. No.: 18/241,482

(22) Filed: Sep. 1, 2023

Related U.S. Application Data
(60) Provisional application No. 63/404,523, filed on Sep. 7, 2022.
(60) Provisional application No. 63/421,011, filed on Oct. 31, 2022.
(60) Provisional application No. 63/524,064, filed on Jun. 29, 2023.

(51) Int. Cl. A61K 31/575; A61K 31/192; A61P 25/28; G01N 33/68

(52) U.S. Cl. CPC A61K 31/575; A61K 31/192; A61P 25/28; G01N 33/6896; G01N 2333/4737; G01N 2800/28; G01N 2800/52

(57) ABSTRACT

Provided herein are methods and compositions for treating at least one symptom of ALS, slowing ALS disease progression, or reducing the deterioration of one or more bodily functions affected by ALS in a subject. The methods can include administering to the subject a bile acid or a pharmaceutically acceptable salt thereof and a phenylbutyrate compound.

### CROSS-REFERENCE TO RELATED APPLICATIONS

This application claims the benefit of priority under 35 U.S.C. §119 to U.S. Provisional Application No. 63/404,523, filed on Sep. 7, 2022, U.S. Provisional Application No. 63/421,011, filed on Oct. 31, 2022, and U.S. Provisional Application No. 63/524,064, filed on Jun. 29, 2023. The entire contents of each of these priority applications are incorporated herein by reference.

### TECHNICAL FIELD

The present disclosure generally relates to compositions and methods for treating Amyotrophic lateral sclerosis.

### BACKGROUND

Amyotrophic lateral sclerosis (ALS) is the most prevalent progressive motor neuron disease. ALS causes the progressive degeneration of motor neurons, resulting in rapidly progressing muscle weakness and atrophy that eventually leads to partial or total paralysis. Median survival from symptom onset is 2 to 3 years, with respiratory failure being the predominant cause of death. ALS treatment currently centers on symptom management. Accordingly, there is a need for improved therapies for treating ALS.

### SUMMARY

The present disclosure provides, in one aspect, methods of reducing the level of YKL-40 or C-reactive protein (CRP) in a biological sample of a human subject, the method comprising: determining or having determined a level of YKL-40 or CRP in a biological sample of a human subject, and administering to the human subject Taurursodiol (TURSO) and sodium phenylbutyrate. In some embodiments, the human subject has one or more symptoms of ALS.

Also provided herein are methods of treating at least one symptom of ALS in a human subject, the method comprising: (a) determining or having determined a first level of YKL-40 in a first biological sample of a human subject, (b) administering to the human subject TURSO and sodium phenylbutyrate, (c) determining or having determined a second level of YKL-40 in a second biological sample of the human subject, and (d) continuing administering to the human subject TURSO and sodium phenylbutyrate if the second level of YKL-40 is higher than the first level of YKL-40.

In another aspect, provided herein are methods of treating at least one symptom of .ALS in a human subject, the method comprising (a) determining or having determined a first level of YKL-40 in a first biological sample of a human subject, (b) administering to the human subject TURSO and sodium phenylbutyrate, (c) determining or having determined a second level of YKL-40 in a second biological sample of the human subject, and (d) continuing administering to the subject TURSO and sodium phenylbutyrate if the second level of YKL-40 is lower than the first level of YKL-40. In some embodiments, step (b) comprises administering to the human subject TURSO and sodium phenylbutyrate for about 24 weeks and step (d) comprises continuing administering to the human subject PASO and sodium phenylbutyrate if the second level of YKL-40 is lower than the first level of YKL-40 by about 0.75 ng/mL or more. In some embodiments, step (b) comprises administering to the human subject TURSO and sodium phenylbutyrate for about 24 weeks and step (d) comprises continuing administering to the human subject TURSO and sodium phenylbutyrate if the second level of YKL-40 is lower than the first level of YKL-40 by about 0.75 ng/mL or less.

Also provided herein are methods of treating at least one symptom of ALS in a human subject, the methods comprising: (a) administering to the human subject TURSO and sodium phenylbutyrate, (b) determining or having determined that a level of YKL-40 in a biological sample of the human subject is higher than about 33 ng/'mL, and (c) continuing administering to the human subject TURSO and sodium phenylbutyrate,

In a further aspect, provided herein are methods of treating at least one symptom of ALS in a human subject, the methods comprising: (a) determining or having determined a first level of C-reactive protein (CRP) in a first biological sample of a human subject, (b) administering to the human subject TURSO and sodium phenylbutyrate, (c) determining or having determined a second level of GRP in a second biological sample of the human subject, and (d) continuing administering to the subject TURSO and sodium phenylbutyrate if the second level of CRP is higher than the first level of CRP.

In another aspect, provided herein are methods of treating at least one symptom of ALS in a human subject, the method comprising: (a) determining or having determined a first level of C-reactive protein (CRP) in a first biological sample of a human subject, (b) administering to the human subject TURSO and sodium phenylbutyrate, (c) determining or having determined a second level of CRP in a second biological sample of the human subject, and (d) continuing administering to the subject TURSO and sodium phenylbutyrate if the second level of CRP is lower than the first level of CRP.

Also provided herein are methods of treating at least one symptom of ALS in a human subject, the methods comprising: (a) administering to the human subject TURSO and sodium phenylbutyrate, (b) determining or having determined that the level of C-reactive protein (CRP) in a biological sample of the subject is higher than about 1835 ng/mL, and (c) continuing administering to the subject TURSO and sodium phenylbutyrate.

In some embodiments of any of the methods described herein, the TURSO is administered at a dose of about 10 mg/kg to about 50 mg/kg of the body weight of the subject. in some embodiments, the sodium phenylbutyrate is administered at a dose of about 10 mg/kg to about 400 mg/kg of the body weight of the subject. In some embodiments, the TURSO and the sodium phenylbutyrate are administered once a day or twice a day. In some embodiments, the TURSO is administered at an amount of about 1 gram once a day or twice a day. In some embodiments, the sodium phenylbutyrate is administered at an amount of about 3 grams once a day or twice a day. In some embodiments, the TURSO and the sodium phenylbutyrate are administered separately. In some embodiments, the TURSO and the sodium phenylbutyrate are administered concurrently. In some embodiments, the TURSO and the sodium phenylbutyrate are administered orally or through a feeding tube. In some embodiments, the TURSO is administered at an amount of about 1 gram once a day or twice a day orally or through a feeding tube, and the sodium phenylbutyrate is administered at an amount of about 3 grams once a day or twice a day orally or through a feeding tube. In some embodiments, the method comprises administering about 1 gram of TURSO once a day and about 3 grams of sodium phenylbutyrate once a day for about 14 days, followed by administering about 1 gram of TURSO twice a day and about 3 grams of sodium phenylbutyrate twice a day. In some embodiments, the TURSO and the sodium phenylbutyrate are formulated as a single powder formulation. in some embodiments, the biological sample is a CSF sample or a serum sample. In some embodiments, the human subject is diagnosed with ALS. In some embodiments, the human subject is suspected as having ALS.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1, shows the treatment-dependent rates of decline in ALSFRS-R total score estimated in the modified intent-to-treat (miff) population in the primary analysis.

FIG. 1B shows the treatment-dependent rates of decline in ALSFRS-R total score estimated in the on-drug population in the primary analysis.

FIG. 2 is a bar graph showing YKL-40 concentration at week 24 in AMX0035 and placebo groups.

FIG. 3 is a graph showing change in YKL-40 plasma concentration relative to baseline over 24 weeks.

### DETAILED DESCRIPTION

Although the precise cause of ALS is unknown, ALS is strongly characterized by nerve cell death and inflammation. Together these processes form a toxic cycle that is a key driver of progressive neurological decline. The present disclosure provides methods of treating at least one symptom of ALS, methods of reducing ALS disease progression; and methods of reducing the deterioration of one or more bodily functions affected by ALS, maintaining one or more bodily functions affected by ALS, or improving one or more bodily functions affected by ALS. For example, provided herein are methods for reducing the CSF or blood (e.g., plasma) levels of YKL-40 in an ALS subject. The methods described herein are also useful in treating or preventing or ameliorating at least one symptom of benign fasciculation syndrome (BFS) or cramp fasciculation syndrome (CFS). The methods include administering a bile acid or a pharmaceutically acceptable salt thereof, and a phenylbutyrate compound.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art.

### I. Amyotrophic Lateral Sclerosis (ALS)

The terms “amyotrophic lateral sclerosis” and “ALS” are used interchangeably herein, and include all of the classifications of ALS known in the art, including, but not limited to classical ALS (e.g., ALS that affects both lower and upper motor neurons), Primary Lateral Sclerosis (PLS, e.g., those that affect only the upper motor neurons), Progressive Bulbar Palsy (PBP or Bulbar Onset, a version of ALS that typically begins with difficulties swallowing, chewing and speaking) and Progressive Muscular Atrophy (PMA, typically affecting only the lower motor neurons). The terms include sporadic and familial (hereditary) ALS, ALS at any rate of progression (e.g., rapid, non-slow or slow progression) and ALS at any stage (e.g., prior to onset, at onset and late stages of ALS).

The subjects in the methods described herein may exhibit one or more symptoms associated with ALS, or have been diagnosed with ALS. In some embodiments, the subjects may be suspected as having ALS, and/or at risk for developing ALS.

The subjects in the methods described herein may exhibit one or more symptoms associated with benign fasciculation syndrome (BFS) or cramp-fasciculation syndrome (CFS).

Some embodiments of any of the methods described herein can further include determining that a subject has or is at risk for developing ALS, diagnosing a subject as having or at risk for developing ALS, or selecting a subject having or at risk for developing ALS. Likewise, some embodiments of any of the methods described herein can further include determining that a subject has or is at risk for developing benign fasciculation syndrome or cramp fasciculation syndrome, diagnosing a subject as having or at risk for developing BFS or CFS, or selecting a subject having or at risk for developing BFS or CFS.

In some embodiments of any of the methods described herein, the subject has shown one or more symptoms of ALS for about 24 months or less (e.g., about 23, 22, 21, 20, 19, 18, 17, 16, 15, 14, 13, 12, 11, 10, 9, 8, 7, 6, 5, 4, 3, 2, 1 month, or 1 week or less). In some embodiments, the subject has shown one or more symptoms of ALS for about 36 months or less (e.g., about 35, 34, 33, 32, 31, 30, 29, 28, 27, 26, or 25 months or less).

The order and type of ALS symptoms displayed by a subject may depend on which motor neurons in the body are damaged first, and consequently which muscles in the body are damaged first. For example, bulbar onset, limb onset, or respiratory onset ALS may present with similar or different symptoms. In general, ALS symptoms may include muscle weakness or atrophy (e.g., affecting upper body, lower body, and/or speech), muscle fasciculation (twitching), cramping, or stiffness of affected muscles. Early symptoms of ALS may include those of the arms or legs, difficulty in speaking clearly or swallowing (e.g., in bulbar onset ALS). Other symptoms include loss of tongue mobility, respiratory difficulties, difficulty breathing or abnormal pulmonary function, difficulty chewing, and/or difficulty walking (e.g., resulting in stumbling). Subjects may have respiratory muscle weakness as the initial manifestation of ALS symptoms. Such subjects may have very poor prognosis and in some instances have a median survival time of about two months from diagnosis. In some subjects, the time of onset of respiratory muscle weakness can be used as a prognostic factor.

ALS symptoms can also be classified by the part of the neuronal system that is degenerated, namely, upper motor neurons or lower motor neurons. Lower motor neuron degeneration manifests, for instance, as weakness or wasting in one or more of the bulbar, cervical, thoracic, and/or lumbosacral regions. Upper motor neuron degeneration can include increased tendon reflexes, spasticity, pseudo bulbar features, Hoffmann reflex, extensor plantar response, and exaggerated reflexes (hyperreflexia) including an overactive gag reflex. Progression of neuronal degeneration or muscle weakness is a hallmark of the disease. Accordingly, some embodiments of the present disclosure provide a method of ameliorating at least one symptom of lower motor neuron degeneration, at least one symptom of upper motor neuron degeneration, or at least one symptom from each of lower motor neuron degeneration and upper motor neuron degeneration. In some embodiments of any of the methods described herein, symptom onset can be determined based on information from subject and/or subject's family members. In some embodiments, the median time from symptom onset to diagnosis is about 12 months.

In some instances, the subject has been diagnosed with ALS. For example, the subject may have been diagnosed with ALS for about 24 months or less (e.g., about 23, 22, 21, 20, 19, 18, 17, 16, 15, 14, 13, 12, 11, 10, 9, 8, 7, 6, 5, 4, 3, 2, or 1 month or less). For example, the subject may have been diagnosed with ALS for 1 week or less, or on the same day that the presently disclosed treatments are administered. The subject may have been diagnosed with ALS for more than about 24 months (e.g., more than about 28, 32, 36, 40, 44, 48, 52, 56, 60, 64, 68, 72, 76, or 80 months). Methods of diagnosing ALS are known in the art. For example, the subject can be diagnosed based on clinical history, family history, physical or neurological examinations((e.g., signs of lower motor neuron or upper motor neuron degeneration). The subject can be confirmed or identified, e.g. by a healthcare professional, as having ALS. Multiple parties may be included in the process of diagnosis. For example, where samples are obtained from a subject as part of a diagnosis, a first party can obtain a sample from a subject and a second party can test the sample. In some embodiments of any of the human subjects described herein, the subject is diagnosed, selected, or referred by a medical practitioner e.g., a general practitioner).

In some embodiments, the subject fulfills the El Escorial criteria for probable or definite ALS, i.e. the subject presents:
1. Signs of lower motor neuron (LMN) degeneration by clinical, electrophysiological or neuropathologic examination;
2. Signs of upper motor neuron (UMN) degeneration by clinical examination; and
3. Progressive spread of signs within a region or to other regions, together with the absence of:

Electrophysiological evidence of other disease processes that might explain the signs of LMN and/or UMN degenerations; and

Neuroimaging evidence of other disease processes that might explain the observed clinical and electrophysiological signs.

Under the El Escorial criteria, signs of LMN and UMN degeneration in four regions are evaluated, including brainstem, cervical, thoracic, and lumbrasacral spinal cord of the central nervous system. The subject may be determined to be one of the following categories:
A. Clinically Definite ALS, defined on clinical evidence alone by the presence of UMN, as well as LMN signs, in three regions.
B. Clinically Probable ALS, defined on clinical evidence alone by UMN and LMN signs in at least two regions with some UMN signs necessarily rostral to (above) the LMN signs.
C. Clinically Probable ALS—Laboratory-supported, defined when clinical signs of UMN and LMN dysfunction are in only one region, or when UMN signs alone are present in one region, and LMN signs defined by EMG criteria are present in at least two limbs, with proper application of neuroimaging and clinical laboratory protocols to exclude other causes.
D. Clinically Possible ALS, defined when clinical signs of UMN and LMN dysfunction are found together in only one region or UMN signs are found alone in two or more regions; or LMN signs are found rostral to UMN signs and the diagnosis of Clinically Probable-Laboratory-supported.

In some embodiments, the subject has clinically definite ALS (e.g., based on the El Escorial criteria).

The subject can be evaluated and/or diagnosed using the Revised Amyotrophic Lateral Sclerosis Functional Rating Scale (ALSFRS-R). The ALSFRS-R is an ordinal rating scale (ratings 0-4) used to determine subjects' assessment of their capability and independence in 12 functional activities relevant in ALS. ALSFRS-R scores calculated at diagnosis can be compared to scores throughout time to determine the speed of progression. Change in ALSFRS-R scores can be correlated with change in strength over time, and can be associated with quality of life measures and predicted survival. ALSFRS-R demonstrates a linear mean slope and can be used as a prognostic indicator (See e.g., Berry et al. Amyotroph Lateral Scler Frontotemporal Degener 15:1-8, 2014; Traynor et al., Neurology 63:1933-1935, 2004; Simon et al., Ann Neurol 76:643-657, 2014; and Moore et al, Amyotroph Lateral Scler Other Motor Neuron Disord 4:42, 2003),

In the ALSFRS-R, functions mediated by cervical, trunk, lumbosacral, and respirator. muscles are each assessed by 3 items. Each item is scored from 0-4, with 4 reflecting no involvement by the disease and 0 reflecting maximal involvement. The item scores are added to give a total. Total scores reflect the impact of ALS, with the following exemplary categorization: >40 (minimal to mild); 39-30 (mild to moderate); <30 (moderate to severe); <20 (advanced disease)

For example, a subject can have an ALSFRS-R score (e.g., a baseline ALSFRS-R score) of 40 or more (e.g., at least 41, 42, 43, 44, 45, 46, 47, or 48), between 30 and 39, inclusive (e.g., 31, 32, 33, 34. 35, 36, 37, or 38), or 30 or less (e.g., 21, 22, 23, 24, 25, 26, 27, 28, or 29). In some embodiments of any of the methods described herein, the subject has an ALSFRS-R score (e.g.; a baseline ALSFRS-R score) of 40 or less (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10 or less). In some embodiments, the subject has an ALSFRS-R score (e.g., a baseline ALSFRS-R score) of 20 or less (e.g., 19, 18, 17, 16, 15, 14, 13. 12, 11, 10, 9, 8, 7, 6, 5 or less).

As ALS is a progressive disease, all patients generally will progress over time. However, a large degree of inter-subject variability exists in the rate of progression, as some subjects die or require respiratory support within months while others have relatively prolonged survival. The subjects described herein may have rapid progression ALS or slow progression ALS. The rate of functional decline in a subject with ALS can be measured by the change in ALSFRS-R score per month. For example, the score can decrease by about 1.02 (±=2.3) points per month.

One predictor of patient progression is the patient's previous rate of disease progression (ΔFS), which can be calculated as: ΔFS=(48−ALSFRS-R, score at the time of evaluation)/duration from onset to time of evaluation (month). The ΔFS score represents the number of ALSFRS-R, points lost per month since symptom onset, and can be a significant predictor of progression and/or survival in subjects with ALS (See e.g., Labra et al. J Neurol Neurosurg Psychiatry 87:628-632, 2016 and Kimura et al. Neurology 66:265-267, 2006). The subject may have a disease progression rate (ΔFS) of about 0.50 or less (e.g., about 0.45, 0.40, 0.35, 0.30, 0.25, 0.20, 0.15, or 0.10 or less); between about 0.50 and about 1.20 inclusive (e.g., about 0.55, 0.60, 0.65, 0.70, 0.75, 0.80, 0.85, 0.90, 0.95, 1.00, 1.05, 1,10, or 1.15); or about 1.20 or greater (e.g., about 1.25, 1.30, 1.35, 1.40, 1.45, 1.50, 1.55, 1.60, 1.75, 1.80, 1.85, 1.90, 1.95, or 2,00 or greater). In some embodiments of any of the methods described herein, the subject can have an ALS disease progression rate (ΔFS) of about 0.50 or greater (e.g., about 0.55, 0.60, 0.65, 0.70, 0,75, 0.80, 0.85, 0.90, 0.95, 1.00, 1.05, 1,10, 1.15, 1,20, 1.25. 1.30, 1.35, 1.40, 1.45, 1.50, 1.55, 1,60, 1.75, 1.80, 1.85, 1.90, 1,95, or 2.00 or greater). However, it should be noted that the ΔFS score is a predictor of patient progression, and may under or overestimate a patient's progression once under evaluation.

In some embodiments, since initial evaluation, the subject has lost on average about 0.8 to about 2 (e.g., about 0.9, 1.0, 1.1, 1.2, 1,3, 1.4, 1.5, 1.6, 1,7, 1.8, or 1.9) ALSFRS-P, points per month over 3-12 months. In some embodiments, the subject has lost on average more than about 1.2 ALSFRS-R points per month over 3-12 months since initial evaluation, The subject may have had a decline of at least 3 points (e.g., at least 4, 6, 8, 10, 12, 14, 16, 20, 24, 28, or 32 points) in ALSFRS-R score over 3-12 months since initial evaluation. In some embodiments, the subject has lost on average about 0.8 to about 2 (e.g., about 0.9, 1.0, 1.1, 1,2, 1,3, 1.4, 1.5, 1.6, 1.7, 1.8, or 1.9) ALSFRS-R points per month over the previous 3-12 months. In some embodiments, the subject has lost on average more than about 1.2 (e.g., more than about 1.5, 1.8, 2,0, 2,5, or 3) ALSFRS-R points per month over the previous 3-12 months.

In some embodiments of any of the methods described herein, the presence or level of a marker in a sample obtained from the subject may be used for ALS diagnosis or prognosis, or to track disease activity and treatment responses. Suitable samples include, for example, cells, tissues, or body fluids (e.g. blood, urine, or cerebral spinal fluid (CSF) samples). For instance, levels of phosphorylated neurofilament heavy subunit (pNF-H) or neurofilament light chain (NfL) in the CSF and/or blood are potential biomarkers for ALS diagnosis, prognosis, or to track disease activity or treatment outcomes. pNF-H and NFLlevels appear to be higher in ALS compared to healthy controls and may correlate with clinical progression rate (See, e.g., De Schaepdryver et al. Journal of Neurology, Neurosurgery & Psychiatry 89:367-373, 2018).

The concentration of pNF-H in the CSF and/or blood of a subject with ALS may significantly increase in the early disease stage. Higher levels of pNF-H in the plasma, serum and/or CSF may be associated with faster ALS progression (e.g., faster decline in ALSFRS-R), and/or shorter survival. pNF-H concentration in plasma may be higher in ALS subjects with bulbar onset than those with spinal onset. In some cases, an imbalance between the relative expression levels of the neurofilament heavy and light chain subunits can be used for ALS diagnosis, prognosis, or tracking disease progression.

Methods of detecting pNF-H and NfL (for example, in the cerebrospinal fluid, plasma, or serum) are known in the art and include but are not limited to, ELISA and Simoa assays (See e.g., Shaw et al. Biochemical and Biophysical Research Communications 336:1268-1277, 2005; Ganesalingam et al. Amyotroph Lateral Scler Frontotemporal Degener 14(2):146-9, 2013; De Schaepdryver et al. Annals of Clinical and Translational Neurology 6(10): 1971-1979, 2019; Wilke et al. Clin Chem Lab Med 57(10):1556-1564, 2019; Poesen et al. Front Neurol 9:1167, 2018; Pawlitzki et al. Front. Neurol, 9:1037, 2018; Gille et al. Neuropathol Appl Neurobiol 45(3):291-304, 2019). Commercialized pNF-H detection assays can also be used, such as those developed by EnCor Biotechnology, BioVendor, and Millipore-EMD. Commercial NfL assay kits based on the Simoa technology, such as those produced by Quanterix can also be used (See, e.g., Thouvenot et al. European Journal of Neurology 27:251-257, 2020). Factors affecting pNF-H and NfL levels or their detection in serum or plasma in relation to disease course may differ from those in CSF. The levels of neurofilament (e.g. pNT-H and/or NfL) in the CSF and serum may be correlated (See, e.g., Wilke et al. Clin Chem Lab Med 57(10):1556-1564, 2019.).

Subjects described herein may have a CSF or blood pNF-H level of about 300 pg/mL or higher (e.g., about 350, 400, 450. 500, 550, 600, 650, 700, 750, 800, 850. 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 20150, 2100, 2150, 2200, 2150, 2300, 235 2400, 2450, 2500, 2550, 2600, 2650, 2700, 2750, 2800, 2850, 2900, 3000, 3200, 3500, 3800, or 4000 pg/mL or higher). In some embodiments, the serum pNF-H level can be about 70 to about 1200 pg/ml, (e.g., about 70 to about 1000, about 70 to about 800, about 80 to about 600, or about 90 to about 400 pg/mL). In some embodiments, the CSF pNF-H level can be about 1000 to about 5000 pg/mL (e.g., about 1500 to about 4000, or about 2000 to about 3000 pg/mL).

The subjects may have a CSF or blood level of NfL of about 50 pg/rnL or higher (e.g., about 60, 70, 80, 90, 100, 110, 120, 130, 140, 150, 160, 170, 180, 190, 200, 210, 220, 230, 240, or pg/mL or higher). In some embodiments, the serum NIL level can be about 50 to about 300 pg/mL, (e.g., about 50 to about 280, about 50 to about 250, about 50 to about 200, about 50 to about 150, about 50 to about 100, about 100 to about 300, about 100 to about 250, about 100 to about 200, about 100 to about 150, about 15(1 to about 300, about 150 to about 250, about 150 to about 200, about 200 to about 300, about 200 to about 250, or about 250 to about 300 pg/mL). In some embodiments, the CSF NIL level can be about 2000 to about 40,000 pg/ml, (e.g., about 2000 to about 35,000, about 2000 to about 30,000, about 2000 to about 25,000, about 2000 to about 20,000, about 2000 to about 15,000, about 2000 to about 10,000, about 2000 to about 8000, about 2000 to about 6000, about 2000 to about 4000, about 4000 to about 40,000, about 4000 to about 35,000, about 4000 to about 30,000, about 4000 to about 25,000, about 4000 to about 20,000, about 4000 to about 15,000, about 4000 to about 10,000, about 4000 to about 8000, about 4000 to about 6000, about 6000 to about 40,000, about 6000 to about 35,000, about 6000 to about 30,000, about 6000 to about 25,000, about 6000 to about 20,000, about 6000 to about 15,000, about 6000 to about 10,000, about 6000 to about 8000, about 8000 to about 40,000, about 8000 to about 35,000, about 8000 to about 30,000, about 8000 to about 25,000, about 8000 to about 20,000, about 8000 to about 15,000, about 8000 to about 10,000, about 10,000 to about 40,000, about 10,000 to about 35,000, about 10,000 to about 30,000. about 10,000 to about 25,000, about 10,000 to about 20,000, about 10,000 to about 15,000, about 15,000 to about 40,000, about 15,000 to about 35,000, about 15,000 to about 30,000, about 15,000 to about 25,000, about 15,000 to about 20,000, about 20,000 to about 40.000, about 20,00(1 to about 35,000, about 20,000 to about 30,000, about 20,000 to about 25,000, about 25,000 to about 40,000, about 25,000 to about 35,000, about 25,000 to about 30,000, about 30,000 to about 40,000, about 30,000 to about 35,000, or about 3:5,000 to about 40,000 pg/mL).

In some embodiments of any of the methods described herein, levels of YKL-40 in the CSF and/or blood (e.g., plasma) can be used as a biomarker for ALS diagnosis, prognosis, or to track disease activity or treatment outcomes, YKL-40 (also known as CHI3L1) also has evidence supporting its relevance in ALS. CSF samples from people with ATS have shown elevation in YKL40 compared to age matched controls (p=0.045). YKL40 has been associated with inflammatory processes in neurodegenerative diseases (Llorens F, Thune K, Tahir W, et al. YKL-40 in the brain and cerebrospinal fluid of neurodegenerative dementias. Moi Neurodegener. 2017 Nov 10;12(1):83. doi: 10.1 186/s13024-017-0226-4). YKL-40 shows a correlation with ALSFRS-R progression as measured by ALSFRS-R slope (p<0.001) (Andres-Benito P, Dominguez R, Colomina M J, et al. YKL40 in sporadic amyotrophic lateral sclerosis: cerebrospinal fluid levels as a prognosis marker of disease progression. Aging (Albany NY). 2018 Sep. 13 ;10(9):2367-2382. doi: 10,18632/aging.101551. Erratum in: Aging (Albany NY). 2021 Oct. 31; 13(2423871).

In some embodiments, after administration with any of the compositions described herein (e.g. TURSO and sodium phenylbutyrate), e.g., for about 12 weeks to about 24 weeks, the subjects have a plasma level of YKL-40 of about 25 ng/mL to about 38 ng/mL (e.g., about 26, 27 28, 29, 30, 30.1, 30.2, 30.3, 30.4, 30.5, 30.6, 30.7, 30.8, 30.9, 31.0, 31.1, 31.2. 31.3, 31.4, 31.5, 31.6, 31.7, 31.8, 31.9, 32, 33, 34, 35, 36, or 37 ng/mL).

Additional biomarkers useful for ALS diagnosis, prognosis, and disease progression monitoring are contemplated herein, including but are not limited to, CSF levels of S100-β, cystatin C, and chitotriosidase (CHIT) (See e.g., Chen et al. BMC Neurol 16:173, 2016). Serum levels of uric acid can be used as a biomarker for prognosing ALS (See e.g., Atassi et al. Neurology 83(19):1719-1725, 2014). Akt phosphorylation can also be used as a biomarker for prognosing ALS (See e.g., WO2012/160563). Urine levels of p75ECD and ketones can be used as a biomarker for ALS diagnosis (See e.g., Shepheard et al. Neurology 88:1137-1.143, 2017). Serum and urine levels of creatinine can also be used as a biomarker. Other useful blood, CSF, neurophysiological, and neuroradiological biomarkers for ALS are described in es., Turner et al. Lancet Neurol 8:94-109, 2009. Any of the markers described herein can be used for diagnosing a subject as having ALS, or determining that a subject is at risk for developing ALS.

A subject may also be identified as having ALS, or at risk for developing ALS, based on genetic analysis. Genetic variants associated with ALS are known in the art (See, e.g., Taylor et al. Nature 539:197-206, 2016; Brown and Al-Chalabi N Engl J Med 377:162-72, 2017; and http://alsod.iop.kcl.ac.uk). Subjects described herein can carry mutations in one or more genes associated with familial and/or sporadic ALS. Exemplary genes associated with ALS include but are not limited to: ANG, TARDBP, VCP, VAPB, SQSTM1, DCTN1, FUS, UNC13A, ATXN2, HNRNPA1, CHCHD10, MOBP, C21ORF2, NEK1, TLBA4A, TBK1, MATR₃, PFN1, UBQLN2, TAF15, OPTN, TDP-43, and DAO. Additional description of genes associated with ALS can be found at Therrien et al. Curr Neurol Neurosci Rep 16:59-71, 2016; Peters et al. J Clin Invest 125:2548, 2015, and Pottier et al, J Neurochem, 138:Suppl 1:32-53, 2016. Genetic variants associated with ALS can affect the ALS progression rate in a subject, the pharmacokinetics of the administered compounds in a subject, and/or the efficacy of the administered compounds for a subject.

The subjects may have a mutation in the gene encoding CuZn-Superoxide Dismutase (SOD1). Mutation causes the SOD1 protein to be more prone to aggregation, resulting in the deposition of cellular inclusions that contain misfolded SOD1 aggregates (See e.g., Andersen et al., Nature Reviews Neurology 7:603-615, 2011), Over 100 different mutations in SOD1 have been linked to inherited ALS, many of which result in a single amino acid substitution in the protein, In some embodiments, the SOD1 mutation is A4V (i.e., a substitution of valine for alanine at position 4). SOD1 mutations are further described in, e.g., Rosen et al. Hum. Mol, Genet. 3, 981-987, 1994 and Rosen et al. Nature 362:59-62, 1993. In some embodiments, the subject has a mutation in the C90RF72 gene. Repeat expansions in the C9ORF72 gene are a frequent cause of ALS, with both loss of function of C9ORF72 and gain of toxic function of the repeats being implicated in ALS (See e.g., Balendra and Isaacs, Nature Reviews Neurology 14:544-558, 2018). The methods described herein can include, prior to administration of a bile acid and a phenylbutyrate compound, detecting a SOD1 mutations and/or a C9ORF72 mutation in the subject. Methods for screening for mutations are well known in the art. Suitable methods include, but are not limited to, genetic sequencing. See, e.g., Hou et al. Scientific Reports 6:32478, 2016; and Vajda et al. Neurology 88:1-9, 2017.

Skilled practitioners will appreciate that certain factors can affect the bioavailability and metabolism of the administered compounds for a subject, and can make adjustments accordingly. These include but are not limited to liver function (e.g. levels of liver enzymes), renal function, and gallbladder function (e.g., ion absorption and secretion, levels of cholesterol transport proteins). There can be variability in the levels of exposure each subject has for the administered compounds (e.g., bile acid and a phenylbutyrate compound), differences in the levels of excretion, and in the pharmacokinetics of the compounds in the subjects being treated. Any of the factors described herein may affect drug exposure by the subject. For instance, decreased clearance of the compounds can result in increased drug exposure, while improved renal function can reduce the actual drug exposure. The extent of drug exposure may be correlated with the subject's response to the administered compounds and the outcome of the treatment.

The subject can be e.g., older than about 18 years of age (e.g., between 18-100, 18-90, 18-80, 18-70, 18-60, 18-50, 18-40, 18-30, 18-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 years of age). The subject can have a BMI of between about 18.5-30 kg/(e.g., between 18.5-28, 18.5-26, 18.5-24, 18.5-22, 18.5-20, 20-30, 20-28, 20-26, 20-24, 20-22, 22-30, 22-28, 22-26, 22-24, 24-30, 24-28, 24-26, 26-30, 26-28, or 28-30 kg/m 2). Having a mutation in any of the ALS-associated genes described herein or presenting with any of the biomarkers described herein may suggest that a subject is at risk for developing ALS. Such subjects can be treated with the methods provided herein for preventative and prophylaxis purposes.

In some embodiments, the subjects have one or more symptoms of benign fasciculation syndrome (BFS) or cramp-fasciculation syndrome (CFS). BFS and CFS are peripheral nerve hyperexcitability disorders, and can cause fasciculation, cramps, pain, fatigue, muscle stiffness, and paresthesia. Methods of identifying subjects with these disorders are known in the art, such as by clinical examination and electromyography.

### II. Composition

The present disclosure provides methods of reducing the level of YKL-40 or C-reactive protein (CRP) in a biological sample of a human subject and method of treating at least one symptom of ALS in a subject, the methods including administering to the subject a bile acid or a pharmaceutically acceptable salt thereof and a phenylbutyrate compound. In some embodiments, the methods include administering a combination of TURSO and sodium phenylbutyrate to the subject.

### Bile Acid

As used herein, “bile acid” refers to naturally occurring surfactants having a nucleus derived from cholanic acid substituted with a 3α-hydroxyl group and optionally with other hydroxyl groups as well, typically at the C6, C7 or C12 position of the sterol nucleus. Bile acid derivatives (e.g., aqueous soluble bile acid derivatives) and bile acids conjugated with an amine are also encompassed by the term “bile acid”. Bile acid derivatives include, but are not limited to, derivatives formed at the carbon atoms to which hydroxyl and carboxylic acid groups of the bile acid are attached with other functional groups, including but not limited to halogens and amino groups. Soluble bile acids may include an aqueous preparation of a free acid form of bile acids combined with one of HCl, phosphoric acid, citric acid; acetic acid, ammonia, or arginine. Suitable bile acids include but are not limited to, taurursodiol (TURSO), ursodeoxycholic acid (MCA), chenodeoxycholic acid (also referred to as “chenodiol” or “chenic acid”), cholic acid, hyodeoxycholic acid, deoxycholic acid, 7-oxolithocholic acid, lithocholic acid, iododeoxycholic acid, iocholic acid, taurochenodeoxycholic acid, taurodeoxycholic acid, glycoursodeoxycholic acid, taurocholic acid, glycocholic acid, or an analog, derivative, or prodrug thereof.

In some embodiments, the bile acids of the present disclosure are hydrophilic bile acids. Hydrophilic bile acids include but are not limited to, TURSO, UDCA, chenodeoxycholic acid, cholic acid, hyodeoxycholic acid, lithocholic acid, and glycoursodeoxycholic acid. Pharmaceutically acceptable salts or solvates of any of the bile acids disclosed herein are also contemplated. In some embodiments, bases commonly employed to form pharmaceutically acceptable salts of the bile acids of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, his-, or tris-(2-OH-(C1-C6)-alkylamine), such as N,N-dimethyl-N-(2-hydroxyethyparnine or tri-(2-hydroxyethvl)amine; N-methyl-D-glucamine; morpholine; thiomorpholine; piperidine; pyrrolidine; and amino acids such as arginine; lysine, and the like.

The terms “tauroursodeoxycholic acid” (TUDCA) and “taurursodiol” (TURSO) are used interchangeably herein.

The bile acid described herein can be TURSO, as shown in formula I (with labeled carbons to assist in understanding where substitutions may be made).

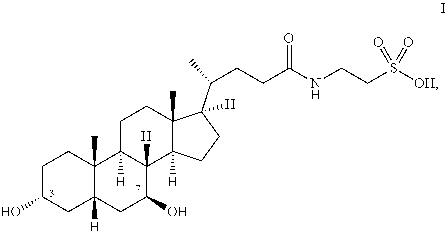

or a pharmaceutically acceptable salt thereof.

The bile acid described herein can be UDCA as shown in formula II (with labeled carbons to assist in understanding where substitutions may be made).

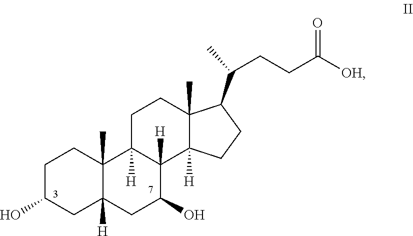

or a pharmaceutically acceptable salt thereof.

Derivatives of bile acids of the present disclosure can be physiologically related bile acid derivatives. For example, any combination of substitutions of hydrogen at position 3 or 7, a shift in the stereochemistry of the hydroxyl group at positions 3 or 7, in the formula of TURSO or UDCA are suitable for use in the present composition.

The “bile acid” can also be a bile acid conjugated with an amino acid. The amino acid in the conjugate can be, but are not limited to, taurine, glycine, glutamine, asparagine, methionine, or carbocysteine. Other amino acids that can be conjugated with a bile acid of the present disclosure include arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid. One example of such a bile acid is a compound of formula III:

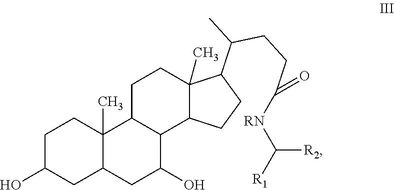

wherein
R is —H or C₁-C₄ alkyl;
R₁ is —CH₂—SO₃R₃, CH₂ COOH, or CH₂CH₂COOH, and R₂ is —H;
or R₁ is —COOH and R₂ is —CH₂—CH₂—CONH₂, —CH₂—CONH₂, —CH₂—CH₂—SCH₃, CH₂CH₂CH₂NH(C═NH)NH₂, CH₂(imidazolyl), CH₂CH₂CH₂CH₂NH2, CH₂COOH, CH₂CH₂COOH:, CH₂OH, CH(OH)CH₃, CH₂SH, pyrrolidin-2-yl, CH₃, 2-propyl, 2-butyl, 2-methyibutyl, CH₂ (henyl), CH₂(4-OH-phenyl), or —CH₂—S—CH₂—COOH and
R₃ is —H or the residue of an amino acid, or a pharmaceutically acceptable analog, derivative, prodrug thereof, or a mixture thereof. One example of the amino acid is a basic amino acid. Other examples of the amino acid include glycine, glutamine, asparagine, methionine, carbocysteine, arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid.

Another example of a bile acid of the present disclosure is a compound of formula IV:

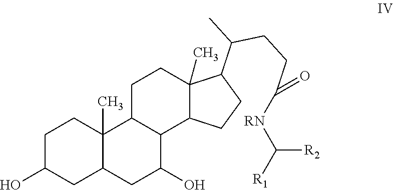

wherein
R is —H or C₁-C₄ alkyl;
R₁ is —CH₂—SO₃R₃, and R₂ is —H;

Is
or R₁ is —COOH and R₂ is —CH₂—CH₂—CONH₂, —CH₂—CONH2, —CH2—CH₂—SCH₃, or —CH₂—S—CH₂—COOH; and
R₃ is —H or the residue of a basic amino acid, or a pharmaceutically acceptable analog, derivative, prodrug thereof, or a mixture thereof. Examples of basic amino acids include lysine, histidine, and arginine.

In some embodiments, the bile acid is TURSO. TURSO is an ambiphilic bile acid and is the taurine conjugate form of UDCA. TURSO recovers mitochondrial bioenergetic deficits through incorporating into the mitochondrial membrane, reducing Bax translocation to the mitochondrial membrane, reducing mitochondrial permeability, and increasing the apoptotic threshold of the cell (Rodrigues et al. Biochemistry 42, 10: 3070-3080, 2003). It is used for the treatment of cholesterol gallstones, where long periods of treatment is generally required (e.g., 1 to 2 years) to obtain complete dissolution. It has been used for the treatment of cholestatic liver diseases including primary cirrhosis, pediatric familial intrahepatic cholestasis and primary sclerosing cholangitis and cholestasis due to cystic fibrosis. TURSO is contraindicated in subjects with biliary tract infections, frequent biliary colic, or in subjects who have trouble absorbing bile acids (e.g. ileal disease or resection). Drug interactions may include with substances that inhibit the absorption of bile acids, such as cholestyramine, and with drugs that increase the elimination of cholesterol in the bile (TURSO reduces biliary cholesterol content). Based on similar physicochemical characteristics, similarities in drug toxicity and interactions exist between TURSO and UDCA. The most common adverse reactions reported with the use of TURSO (≥1%) are: abdominal discomfort, abdominal pain, diarrhea, nausea, pruritus, and rash. There are some cases of pruritus and a limited number of cases of elevated liver enzymes.

In some embodiments, the bile acid is UDCA. UDCA, or ursodiol, has been used for treating gallstones, and is produced and secreted endogenously by the liver as a taurine (TURSO) or glycine (GUDCA) conjugate. Taurine conjugation increases the solubility of UDCA by making it more hydrophilic. TURSO is taken up in the distal ileum under active transport and therefore likely has a slightly a longer dwell time within the intestine than UDCA which is taken up more proximally in the ileum. Ursodiol therapy has not been associated with liver damage. Abnormalities in liver enzymes have not been associated with Actigall® (Ursodiol USP capsules) therapy and, Actigall® has been shown to decrease liver enzyme levels in liver disease. However, subjects given Actigall® should have SGOT (AST) and SGPT (ALT) measured at the initiation of therapy and thereafter as indicated by the particular clinical circumstances. Previous studies have shown that bile acid sequestering agents such as cholestyramine and colestipol may interfere with the action of ursodiol by reducing its absorption. Aluminum-based antacids have been shown to adsorb bile acids in vitro and may be expected to interfere with ursodiol in the same manner as the bile acid sequestering agents. Estrogens, oral contraceptives, and clofibrate (and perhaps other lipid-lowering drugs) increase hepatic cholesterol secretion, and encourage cholesterol gallstone formation and hence may counteract the effectiveness of ursodiol.

### Phenylbutyrate Compounds

Phenylbutyrate compound is defined herein as encompassing phenylbutyrate (a low molecular weight aromatic carboxylic acid) as a free acid (4-phenylbutyrate (4-PBA), 4-phenylbutyric acid, or phenylbutyric acid), and pharmaceutically acceptable salts, co-crystals, polymorphs, hydrates, solvates, conjugates, derivatives or pro-drugs thereof. Phenylbutyrate compounds described herein also encompass analogs of 4-PBA, including but not limited to Glyceryl Tri-(4-phenylbutyrate), phenylacetic acid (which is the active metabolite of PBA), 2-(4-Methoxyphenoxy) acetic acid (2-POA-OMe), 2-(4-Nitrophenoxy) acetic acid (2-POAA-NO2), and 2-(2-Naphthyloxy) acetic acid (2-NOAA), and their pharmaceutically acceptable salts: Phenylbutyrate compounds also encompass physiologically related 4-PBA species, such as but not limited to any substitutions for Hydrogens with Deuterium in the structure of 4-PBA. Other HDAC₂ inhibitors are contemplated herein as substitutes for phenylbutyrate compounds.

Physiologically acceptable salts of phenylbutyrate, include, for example sodium, potassium, magnesium or calcium salts. Other example of salts include ammonium, zinc, or lithium salts, or salts of phenylbutyrate with an orgain amine, such as lysine or arginine.

In some embodiments of any of the methods described herein, the phenylbutyrate compound is sodium phenylbutyrate. Sodium phenylbutyrate has the following formula:

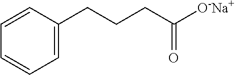

Phenylbutyrate is a pan-HDAC inhibitor and can ameliorate ER stress through upregulation of the master chaperone regulator DJ-1 and through recruitment of other chaperone proteins (See e.g., Zhou et al. J Biol Chem. 286: 14941-14951, 2011 and Suaud et al. JBC. 286:21239-21253, 2011). The large increase in chaperone production reduces activation of canonical Eft stress pathways, folds misfolded proteins, and has been shown to increase survival in in vivo models including the G₃₉₃A SODI mouse model of ALS (See e.g., Ryu, H et al. J Neurochem. 93:1087-1098, 2005).

In some embodiments, the combination of a bile acid (e.g., TURSO), or a pharmaceutically acceptable salt thereof, and a phenylbutyrate compound (e.g., sodium phenylbutyrate) has synergistic efficacy when dosed in particular ratios (e.g., any of the ratios described herein), in treating one or more symptoms associated with ALS. The combination can, for example, induce a mathematically synergistic increase in neuronal viability in a strong oxidative insult model (H₂O₂-mediated toxicity) by linear modeling, through the simultaneous inhibition of endoplasmic reticulum stress and mitochondrial stress (See, e.g. U.S. Pat. No. 9,872,865 and U.S. Pat. No. 10,251,896).

### Formulation

Bile acids and phenylbutyrate compounds described herein can be formulated for use as or in pharmaceutical compositions. For example, the methods described herein can include administering an effective amount of a composition comprising TURSO and sodium phenylbutyrate. The term “effective amount”, as used herein, mfer to an amount or a concentration of one or more drugs for a period of time (including acute or chronic administration and periodic or continuous administration) that is effective within the context of its administration for causing an intended effect or physiological outcome. TURSO and sodium phenylbutyrate can be formulated as a single dosage form. For example ; TURSO and sodium phenylbutyrate can be formulated as a single power formulation. The formulation can include about 5% to about 15% www (e.g., about 6% to about 14%, about 7% to about 13%, about 8% to about 12%, about 8% to about 11%, about 9% to about 10%m or about 9.7% w/w) of TURSO and about 15% to about 45% w/w (e.g., about 20% to about 40%, about 25% to about 35%, about 28% to about 32%, or about 29% to about 30%, e.g., about 29.2% w/w) of sodium phenylbutyrate. In some embodiments, the composition includes about 9.7% w/w of TURSO and 29.2% w/w of sodium phenylbutyrate.

The sodium phenylbutyrate and TURSO can be present in the composition at a ratio by weight of between about 1:1 to about 4:1 (e.g., about 2:1 or about 3:1). In some embodiments, the ratio between sodium phenylbutyrate and MRSO is about 3:1.

TURSO and sodium phenylbutyrate can also be formulated as separate dosage forms. For example, TURSO and sodium phenylbutyrate can be formulated as separate dosage forms and administered separately or together, including as part of a regimen of treatment. The compositions described herein can include any pharmaceutically acceptable carrier, adjuvant, and/or vehicle. The term “pharmaceutically acceptable carrier or adjuvant” refers to a carrier or adjuvant that may be administered to a patient, together with a compound disclosed herein, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

Compositions of the present disclosure can include about 8% to about 24% w/w of dextrates (e.g., about 9% to about 23%, about 10% to about 22%, about 10% to about 20%, about 11% to about 21%, about 12% to about 20%, about 13% to about 19%, about 14% to about 18%, about 14% to about 17%, about 15% to about 16%, or about 15.6% w/w of dextrates). Both anhydrous and hydrated dextrates are contemplated herein. The dextrates of the present disclosure can include a mixture of saccharides developed from controlled enzymatic hydrolysis of starch. Some embodiments of any of the compositions described herein include hydrated dextrates (e.g., NF grade, obtained from JRS Pharma, Colonial Scientific, or Quadra).

Compositions of the present disclosure can include about 1% to about 6% w/w of sugar alcohol (e.g., about 2% to about 5%, about 3% to about 4%, or about 3.9% w/w of sugar alcohol). Sugar alcohols can be derived from sugars and contain one hydroxyl group (—OH) attached to each carbon atom. Both disaccharides and monosaccharides can form sugar alcohols. Sugar alcohols can be natural or produced by hydrogenation of sugars. Exemplary sugar alcohols include but are not limited to, sorbitol, xylitol, and mannitol. In some embodiments, the composition comprises about 1% to about 6% w/w (e.g., about 2% to about 5%, about 3% to about 4%, or about 3.9% w/w) of sorbitol.

Compositions of the present disclosure can include about 22% to about 35% w/w of maltodextrin (e.g., about 22% to about 33%, about 24% to about 31%, about 25% to about 32%, about 26% to about 30%, or about 28% to about 29% w/w, e.g., about 28,.3% w/w of maltodextrin). Maltodextrin can form a flexible helix enabling the entrapment of the active ingredients (e.g., any of the phenylbutyrate compounds and bile acids described herein) when solubilized into solution, thereby masking the taste of the active ingredients. Maltodextrin produced from any suitable sources are contemplated herein, including but not limited to, pea, rice, tapioca, corn, and potato. In some embodiments, the maltodextrin is pea maltodextrin. In some embodiments, the composition includes about 28.3% w/w of pea maltodextrin. For example, pea maltodextrin obtained from Roquette (KLEPTOSE® LINECAPS) can be used.

The compositions described herein can further include sugar substitutes (e.g. sucralose). For example, the compositions can include about 0.5% to about 5% w/w of sucralose (e.g., about 1% to about 4%, about 1% to about 3%, or about 1% to about 2%, e.g., about 1.9% w/w of sucralose). Other sugar substitutes contemplated herein include but are not limited to aspartame, neotame, acesulfame potassium, saccharin, and advantame.

In some embodiments, the compositions include one or more flavorants. The compositions can include about 2% to about 15% w/w of avorants (e.g., about 3% to about 13%, about 3% to about 12%, about 4% to about 9%, about 5% to about 10%, or about 5% to about 8%, e.g., about 7.3% w/w). Flavorants can include substances that give another substance flavor, or alter the characteristics of a composition by affecting its taste. Flavorants can be used to mask unpleasant tastes without affecting physical and chemical stability, and can be selected based on the taste of the drug to be incorporated. Suitable flavorants include but are not limited to natural flavoring substances, artificial flavoring substances, and imitation flavors. Blends of flavorants can also be used. For example, the compositions described herein can include two or more (e.g., two, three, four, five or more) flavorants. Flavorants can be soluble and stable in water. Selection of suitable flavorants can be based on taste testing. For example, multiple different flavorants can be added to a composition separately, which are subjected to taste testing. Exemplary flavorants include any fruit flavor powder (e.g., peach, strawberry, mango, orange, apple, grape, raspberry, cherry or mixed berry flavor powder). The compositions described herein can include about 0.5% to about 1.5% w/w e.g., about 1% w/w) of a mixed berry flavor powder and/or about 5% to about 7% w/w (e.g., about 6.3% w/w) of a masking flavor. Suitable masking flavors can be obtained from e.g., Firmenich.

The compositions described herein can further include silicon dioxide (or silica). Addition of silica to the composition can prevent or reduce agglomeration of the components of the composition. Silica can serve as an anti-caking agent, adsorbent, disintegrant, or glidant. In some embodiments, the compositions described herein include about 0.1% to about 2% w/w of porous silica (e.g., about 0.3% to about 1.5%, about 0.5% to about 1.2%, or about 0.8% to about 1%, e.g., 0.9% w/w). Porous silica may have a higher H₂O absorption capacity and/or a higher porosity as compared to fumed silica, at a relative humidity of about 20% or higher (e.g., about 25%, 30%, 35%, 40%, 45%, 50%, 55%, 60%, 65%, 70%, 75%, 80%, 85%, 90%, or 95% or higher). The porous silica can have an H₂O absorption capacity of about 5% to about 40% (e.g. about 20% to about 40%, or about 30% to about 40%) by weight at a relative humidity of about 50%. The porous silica can have a higher porosity at a relative humidity of about 20% or higher (e.g., about 30%, 40%, 50%, 60%, 70%, 80%, 90% or higher) as compared to that of fumed silica. In some embodiments, the porous silica have an average particle size of about 2 μm to about 10 μm (e.g. about 3 μm to about 9 μm, about 4 μm to about 8 μm, about 5 μm to about 8 μm, or about 7.5 μm). In some embodiments, the porous silica have an average pore volume of about 0.1 cc/gm to about 2.0 cc/gm (e.g., about 0.1 cc/gm to about 1.5 cc/gm, about 0.1 cc/gm to about 1 cc/gm, about 0.2 cc/gm to about 0.8 cc/gm, about 0.3 cc/gm to about 0.6 cc/gm, or about 0.4 cc/gm). In some embodiments, the porous silica have a bulk density of about 50 g/L to about 700 DI: (e.g. about 100 g/L to about 600 g/L, about 200 g/L to about 600 g/L, about 400 g/L to about 600 g/L, about 500 g/L to about 600 g/L., about 540 g/L to about 580 g/L, or about 560 g/L). In. some embodiments, the compositions described herein include about 0.05% to about 2% w/w (e.g., any subranges of this range described herein) of Syloid® 63FP (WR Grace).

The compositions described herein can further include one or more buffering agents. For example, the compositions can include about 0.5% to about 5% w/w of buffering agents (e.g., about 1% to about 4% w/w, about 1.5% to about 3.5% w/w, or about 2% to about 3% w/w, e.g. about 2.7% w/w of buffering agents). Buffering agents can include weak acid or base that maintain the acidity or pH of a composition near a chosen value after addition of another acid or base. Suitable buffering agents are known in the art. In some embodiments, the buffering agent in the composition provided herein is a phosphate, such as a sodium phosphate (e.g., sodium phosphate dibasic anhydrous). For example, the composition can include about 2.7% w/w of sodium phosphate dibasic.

The compositions can also include one or more lubricants. For example, the compositions can include about 0.05% to about 1% w/w of lubricants (e.g., about 0.1% to about 0.9%, about 0.2% to about 0.8 about 0.3% to about 0.7%, or about 0.4% to about 0.6%, e.g. about 0.5% w/w of lubricants). Exemplary lubricants include, but are not limited to sodium stearyl fumarate, magnesium stearate, stearic acid, metallic stearates, talc, waxes and glycerides with high melting temperatures, colloidal silica, polyethylene glycols, alkyl sulphates, glyceryl behenate, and hydrogenated oil. Additional lubricants are known in the art. In some embodiments, the composition includes about 0.05% to about 1% w/w (e.g., any of the subranges of this range described herein) of sodium stearyl fumarate. For example, the composition can include about 0.5% w/w of sodium stearyl fumarate.

In some embodiments, the composition include about 29.2% w/w of sodium phenylbutyrate, about 9.7% w/w of PASO, about 15.6% w/w of dextrates, about 3.9% w/w of sorbitol, about 1.9% w/w of sucralose, about 28.3% w/w of maltodexttin, about 7.3% w/w of flavorants, about 0.9% w/w of silicon dioxide, about 2.7% w/w of sodium phosphate (e.g. sodium phosphate dibasic), and about 0.5% w/w of sodium stearyl fumerate.

The composition can include about 3000 mg of sodium phenylbutyrate, about 1000 mg of TURSO, about 1600 mg of dextrates, about 400 mg of sorbitol, about 200 mg of sucralose, about 97.2 mg of silicon dioxide, about 2916 mg of maltodextrin, about 746 mg of flavorants (e.g. about 20 102 mg of mixed berry flavor and about 644 mg of masking flavor), about 280 mg of sodium phosphate (e.g. sodium phosphate dibasic), and about 48.6 mg of sodium stearyl fumerate.

Additional suitable sweeteners or taste masking agents can also be included in the compositions, such as but not limited to, xylose, ribose, glucose, mannose, galactose, fructose, dextrose, sucrose, maltose, steviol glycosides, partially hydrolyzed starch, and corn syrup solid. Water soluble artificial sweeteners are contemplated herein, such as the soluble saccharin salts (e.g., sodium or calcium saccharin salts), cyclamate salts, acesulfam potassium (acesulfame K), and the free acid form of saccharin and aspartame based sweeteners such as L-aspartyl-phenylalanine methyl ester, Alitame® or Neotame®. The amount of sweetener or taste masking agents can vary with the desired amount of sweeteners or taste masking agents selected for a particular final composition.

Pharmaceutically acceptable binders in addition to those described above are also contemplated. Examples include cellulose derivatives including microcrystalline cellulose, low-substituted hydroxypropyl cellulose (e.g. LH 22, LH 21, LH 20, LH 32, LH 31, LH30), starches, including potato starch; croscarmellose sodium (i.e. cross-linked carboxymethylcellulose sodium salt; e.g. Ac-Di-Sol®); alginic acid or alginates; insoluble polyvinylpyrrolidone (e.g. Polyvidon® CL, Polyvidon® CL-M, Kollidon® CL, Polyplasdone® XL, Polyplasdone® XL-10); and sodium carboxymethyl starch (e.g. Primogel® and Explotab®).

Additional tillers, diluents or binders may be incorporated such as polyols, sucrose, sorbitol, mannitol, Erythritol®, Tagatose®, lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avicel® PH105, Elcerna® P100, Emcocel®, Vivacel®, Ming Tai® and Solka-Floc®), hydroxypropylcellulose, L-hydroxypropylcellulose (low-substituted) (e.g. L-HPC—CE131, L-HPC-LH11, LH 22, LH 21, LH 20, LH 32, LH 31, LH30), dextrins, maltodextrins (e.g. Lodex® 5 and Lodex® 10), starches or modified starches (including potato starch, maize starch and rice starch), sodium chloride, sodium phosphate, calcium sulfate, and calcium carbonate.

The compositions described herein can be formulated or adapted for administration to a subject via any route (e.g. any route approved by the Food and Drug Administration (FDA)). Exemplary methods are described in the FDA's CDER Data Standards Manual, version number 004 (which is available at fda.give/cder/dsm/DRG/drg00301.html).

Pharmaceutical compositions are typically formulated to be compatible with its intended route of administration. Examples of routes of administration include parenteral (subcutaneous, intracutaneous, intravenous, intradermal, intramuscular, intra-articular, intraarterial, intrasynovial, intrasternal, intrathecal, intralesional and intracranial injection or infusion techniques), oral (e.g., inhalation or through a feeding tube), transdermal (topical), transmucosal, and rectal administration.

Pharmaceutical compositions can be in the form of a solution or powder for inhalation and/or nasal administration. In some embodiments, the pharmaceutical composition is formulated as a powder filled sachet. Suitable powders may include those that are substantially soluble in water. Pharmaceutical compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions can be orally administered in any orally acceptable dosage form including, but not limited to, powders, capsules, tablets, emulsions and aqueous suspensions, dispersions and solutions. In the case of powders for oral administration, the powders can be substantially dissolved in water prior to administration. In the case of tablets for oral use, carriers which are commonly used include lactose and corn starch. Lubricating agents, such as magnesium stearate, may be added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, the compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some embodiments, therapeutic compositions disclosed herein can be formulated for sale in the US, imported into the US, and/or exported from the US. The pharmaceutical compositions can be included in a container, pack, or dispenser together with instructions for administration. In some embodiments, the invention provides kits that include the bile acid and phenylbutyrate compounds. The kit may also include instructions for the physician and/or patient, syringes, needles, box, bottles, vials, etc.

### II. Methods of Treatment

Provided herein are methods of reducing the level of YKL-40 or C-reactive protein (CRP) in a biological sample of a human subject, the methods comprising determining or having determined a level of YKL-40 or CRP in a biological sample (e.g. a CSF or blood sample) of a human subject, and administering to the human subject TURSO and sodium phenylbutyrate. Iii some embodiments, the subject has one or more symptoms of ALS, has ALS, or is at risk for developing ALS.

In some embodiments, provided herein are methods of treating at least one symptom of ALS in a human subject, the method comprising determining or having determined a level of YKL-40 or CRP in a biological sample of a human subject, and administering to the human subject a combination of a bile acid (e.g. TURSO) and a phenylbutyrate compound (e.g. sodium phenylbutyrate). For example, the methods can include administering about 1 gram of TURSO and about 3 grams of sodium phenylbutyrate once a day for a period of time (e.g., about 14 days), followed by administering about 1 gram of TURSO and about 3 grams of sodium phenylbutyrate twice a day.

The present disclosure also provides methods of treating ALS in a subject, ameliorating at least one symptom of ALS in a subject, or prophylactically treating a subject at risk for developing ALS (e.g., a subject with a family history of ALS) or a subject suspected to be developing ALS (e.g., a subject displaying at least one symptom of ALS, a symptom of upper motor neuron degeneration, and/or a symptom of lower motor neuron degeneration, but not enough symptoms at that time to support a full diagnosis of ALS).

Also provided are methods of ameliorating at least one symptom of lower motor neuron degeneration, at least one symptom of upper motor neuron degeneration, or at least one symptom from each of lower motor neuron degeneration and upper motor neuron degeneration in a subject.

Some embodiments of the present disclosure provide methods of slowing ALS disease progression (e.g., reducing the ALS disease progression rate); and methods of reducing deterioration of muscle strength, respiratory muscle/pulmonary function and/or fine motor skill, as well as methods of maintaining or improving muscle strength, respiratory musclelpulmonary function and/or fine motor skill.

This disclosure further provides methods of treating at least one symptom of bulbar-onset ALS in a human subject. Also provided are methods of ameliorating at least one symptom of benign fasciculation syndrome or cramp fasciculation syndrome.

### Administration of TURSO and Sodium Phenylbutyrate

The methods described herein include administering to the subject a bile acid or pharmaceutically acceptable salt thereof, and a phenylbutyrate compound. The bile acid or a pharmaceutically acceptable salt thereof and the phenylbutyrate compound can be administered separately or concurrently, including as a part of a regimen of treatment. The compounds can be administered daily (e.g. once a day, twice a day, or three times a day or more), weekly, monthly, or quarterly. The compounds can be administered over a period of weeks, months, or years. For example, the compounds can be administered over a period of at least or about 1 week, 2 weeks, 3 weeks, 1 month, 2 months, 3 months, 4 months, 5 months, 6 months, 7 months, 8 months. 9 months, 10 months, 11 months, 1 year, 2 years, 3 years, 4 years, or at least or about 5 years, or more. The compounds can be administered once a day or twice a day for 60 days or less (e.g., 55 days, 50 days, 45 days, 40 days, 35 days, 30 days or less). Alternatively, the bile acid and phenylbutyrate compound can be administered once a day or twice a day for more than 60 days (e.g., more than 65, 70, 75, 80, 85, 90, 95. 100, 105, 110, 115, 120, 130, 140, 150, 160, 180, 200, 250, 300, 400, 500, 600 days).

In some embodiments, the methods provided herein include administering an effective amount of a composition comprising about 1 gram of TURSO and about 3 grams of sodium phenylbutyrate. TURSO can be administered at an amount of about 0.5 to about 5 grams per day (e.g., about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 5, about 1 to about 4.5, about 1 to about 4, about 1 to about 3.5, about 1 to about 3, about 1 to about 2.5, about 1 to about 2, about 1 to about 1.5, about 1.5 to about 5, about 1.5 to about 30 4.5. about 1.5 to about 4, about 1.5 to about 3.5, about 1.5 to about 3, about 1.5 to about 2.5, about 1.5 to about 2, about 2 to about 5, about 2 to about 4.5, about 2 to about 4, about 2 to about 3.5, about 2 to about 3, about 2 to about 2.5, about 2.5 to about 5, about 2.5 to about 4.5, about 2.5 to about 4, about 2.5 to about 3.5, about 2.5 to about 3, about 3 to about 5, about 3 to about 4.5, about 3 to about 4, about 3 to about 3.5, about 3.5 to about 5 about 3.5 to about 4.5, about 3.5 to about 4, about 4 to about 5, about 4 to about 4.5, or about 4.5 to about 5 grams). In some embodiments, TURSO is administered at an amount of about 1 to about 2 grams per day, inclusive (e.g., about 1 to about 1.8 grams, about 1 to about 1.6 grams, about 1 to about 1.4 grams, about 1 to about 1.2 grams, about 1.2 to about 2.0 grams, about 1.2 to about 1.8 grams, about 1.2 to about 1.6 grams, about 1.2 to about 1.4 grams, about 1.4 to about 2.0 grams, about 1.4 to about 1.8 grams, about 1.4 to about 1.6 grams, about 1.6 to about 2.0 grams, about 1.6 to about 1.8 grams, about 1.8 to about 2.0 grams). In some embodiments, RASO is administered at an amount of about I gram per day.

In some embodiments, TURSO is administered at an amount of about 2 grams per day. For example, RASO can be administered at an amount of about 1 gram twice a day.

Sodium phenylbutyrate can be administered at an amount of about 0.5 to about 10 grams per day (e.g., about 1 to about 10, about 1 to about 9, about 1 to about 8, about 1 to about 7, about 1 to about 6, about Ito about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2.5 to about 9.5, about 2.5 to about 8.5, about 2.5 to about 7.5, about 2.5 to about 6.5, about 2.5 to about 5.5, about 2.5 to about 4.5, about 3 to about 10, about 3 to about 9, about 3 to about 8, about 3 to about 7, about 3 to about 6.5, about 3 to about 6, about 3 to about 5, about 4 to about 10, about 4 to about 9, about 4 to about 8, about 4 to about 7, about 4 to about 6, about 5 to about 10, about 5 to about 9, about 5 to about 8, about 5 to about 7, about 6 to about 10, about 6 to about 9, about 6 to about 8, about 7 to about 10, about 7 to about 9, about 8 to about 10 grams per day), In some embodiments, sodium phenylbutyrate is administered at an amount of about 3 to about 6 grams per day, inclusive (e.g., about 3 to about 5.5 grams, about 3 to about 5.0 grams, about 3 to about 4.5 grams, about 3 to about 4.0 grams, about 3 to about 3.5 grams, about 3.5 to about 6 grams, about 3.5 to about 5.5 grams, about 3.5 to about 5.0 grams, about 3.5 to about 4.5 grams, about 3.5 to about 4.0 grams, about 4.0 to about 6 grams, about 4.0 to about 5.5 grams, about 4.0 to about 5.0 grams, about 4.0 to about 4.5 grams, about 4.5 to about 6 grams, about 4.5 to about 5.5 grams, about 4.5 to about 5.0 grams, about 5.0 to about 6 grams, about 5.0 to about 5.5 grams, or about 5.5 to about 6.0 grams). In some embodiments, sodium phenylbutyrate is administered at an amount of about 3 grams per day. In some embodiments, sodium phenylbutyrate is administered at an amount of about 6 grams per day. For example, sodium phenylbutyrate can be administered at an amount of about 3 grams twice a day. In some embodiments, the bile acid and phenylbutyrate compound are administered at a ratio by weight of about 2.5:1 to about 3.5:1 (e.g., about 3:1).

The methods described herein can include administering about 1 gram of TURSO once a day and about 3 grams of sodium phenylbutyrate once a day, or about 1 gram of TURSO twice a day and about 3 grams of sodium phenylbutyrate twice a day. The methods can include administering about 1 gram of TURSO once a day and about 3 grams of sodium phenylbutyrate once a day for at least 14 days (e.g., at least 15, 16, 17, 18, 19, 20, 21, 22, 23, 24, 27, 30, 35, or 40 days), followed by administering about 1 gram of TURSO twice a day and about 3 grams of sodium phenylbutyrate twice a day or at least a day (e.g. at least 30, 40, 50, 60, 80, 100, 120, 150, 180, 250, 300, or 400 days). For example, the methods can include administering about 1 gram of TURSO once a day and about 3 grams of sodium phenylbutyrate once a day for 14-21 days, followed by administering about 1 gram of TURSO twice a day and about 3 grams of sodium phenylbutyrate twice a day.

In some embodiments, the methods described herein include administering to a subject about 10 mg/kg to about 50 mg/kg of body weight of MRS° per day (e.g., about 10 mg/ cg to about 48 mg/kg, about 10mg/kg to about 46 mg/kg, about 10mg/kg to about 44 mg/kg, about 10 mg/kg to about 42 mg/kg, about 10 mg/kg to about 40 mg/kg, about 10 mg/kg to about 38 mg/kg, about 10 mg/kg to about 36 mg/kg, about 10 mg/kg to about 34 mg/kg, about 10 mg/kg to about 32 mg/kg, about 10 mg/kg to about 30 mg/kg, about 10 mg/kg to about 28 mg/kg, about 10 mg/kg to about 2.6 mg/kg, about 10 mg/kg to about 24 mg/kg, about 10 mg/kg to about 22 mg/kg, about 10 mg/kg to about 20 mg/kg, about 10 mg/kg to about 18 mg/kg, about 10 mg/kg to about 16 mg/kg, about 10 mg/kg to about 14 mg/kg , about 10 mg/kg to about 12 mg/kg, about 12 mg/kg to about 50 mg/kg, about 12 mg/kg to about 48 mg/kg, about 12 mg/kg to about 46 mg/kg, about 12 mg/kg to about 44 mg/kg, about 12 mg/kg to about 42 mg/kg, about 12 mg/kg to about 40 ing/kg, about 12 mg/kg to about 38 mg/kg, about 12 mg/kg to about 36 mg/kg, about 12 mg/kg to about 34 mg/kg, about 12 mg/kg to about 32 mg/kg, about 12 mg/kg to about 30 mg/kg, about 12 mg/kg to about 2.8 mg/kg, about 12 mg/kg to about 26 mg g, about 12 mg/kg to about 24 mg/kg, about 12 mg/kg to about 22 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 50 mg/kg, about 14 mg/kg to about 48 mg/kg, about 14 mg/kg to about 46 mg/kg, about 14 mg/kg to about 44 mg/kg, about 14 mg/kg to about 42 mg/kg, about 14 mg/kg to about 40 mg/kg, about 14 mg/kg to about 38 mg/kg, about 14 mg/kg to about 36 mg/kg, about 14 mg/kg to about 34 mg/kg, about 14 mg/kg to about 32 mg/kg, about 14 mg/kg to about 30 mg/kg, about 14 mg/kg to about 28 mg/kg, about 14 mg/kg to about 26 mg/kg, about 14 mg/kg to about 24 mg/kg, about 14 mg/kg to about 2.2 mg/kg, about 14mg/kg to about 20 mg/kg, about 14 mg/kg to about 18 mg/kg, about 14 mg/kg to about 16 mg/kg, about 16 mg/kg to about 50 mg/kg, about 16 mg/kg to about 48 mg/kg, about 16 mg/kg to about 46 mg/kg, about 16 mg/kg to about 44 mg/kg, about 16 mg/kg to about 42 mg/kg, about 16 mg/kg to about 40 mg/kg, about 16 mg/kg to about 38 mg/kg, about 16 mg/kg to about 36 mg/kg, about 16 mg/kg to about 34 mg/kg, about 16 mg/kg to about 32 mg/kg, about 16 mg/kg to about 30 mg/kg, about 16 mg/kg to about 28 mg/kg, about 16 mg/kg to about 26 mg/kg, about 16 mg/kg to about 24 mg/kg, about 16 mg/kg to about 22 mg/kg, about 16 mg/kg to about 20 mg/kg, about 16 mg/kg to about 18 mg/kg, about 18 mg/kg to about 50 mg/kg, about 18 mg/kg to about 48 mg/kg, about 18 mg/kg to about 46 mg/kg, about 18 mg/kg to about 44 mg/kg, about 18 mg/kg to about 42 mg/kg, about 18 mg/kg to about 40 mg/kg, about 18 mg/kg to about 38 mg/kg, about 18 mg/kg to about 36 mg/kg, about 18 mg/kg to about 34 mg/kg, about 18 mg/kg to about 32 nag/kg, about 18 mg/kg to about 30 mg/kg, about 18 mg/kg to about 28 mg/kg, about 18 mg/kg to about 26 mg/kg, about 18 mg/kg to about 24 mg/kg, about 18 mg/kg to about 22 mg/kg, about 18 mg/kg to about 20 mg/kg, about 20 mg/kg to about 50 mg/kg, about 20 mg/kg to about 48 mg/kg, about 20 mg/kg to about 46 mg/kg, about 20 mg/kg to about 44 mg/kg, about 20 mg/kg to about 42 mg/kg, about 20 mg/kg to about 40 mg/kg, about 20 mg/kg to about 38 mg/kg, about 20 mg/kg to about 36 mg/kg, about 20 mg/kg to about 34 mg/kg, about 20 mg/kg to about 32 mg/kg, about 20 mg/kg to about 30 mg/kg, about 20 mg/kg to about 2.8 mg/kg, about 20 mg/kg to about 26 mg/kg, about 20 mg/kg to about 24 mg/kg, about 20 mg/kg to about 22 mg/kg, about 22 mg/kg to about 50 mg/kg, about 22 mg/kg to about 48 mg/kg, about 22 mg/kg to about 46 mg/kg, about 22 mg/kg to about 44 mg/kg, about 22 mg/kg to about 42 mg/kg, about 22 mg/kg to about 40 mg/kg, about 22 mg/kg to about 38 mg/kg, about 22 mg/kg to about 36 mg/kg, about 22 mg/kg to about 34 mg/kg, about 22 mg/kg to about 32 mg/kg, about 2.2 mg/kg to about 30 mg/kg, about 22 mg/kg to about 28 mg/kg, about 22 mg/kg to about 26 mg/kg, about 22 mg/kg to about 24 mg/kg, about 24 mg/kg to about 50 mg/kg, about 24 mg/kg to about 48 mg/kg, about 24 mg/kg to about 46 mg/kg, about 24 mg/kg to about 44 mg/kg, about 24 mg/kg to about 42 mg/kg, about 24 mg/kg to about 40 mg/kg, about 24 mg/kg to about 38 mg/kg, about 24 mg/kg to about 36 mg/kg, about 24 mg/kg to about 34 mg/kg, about 24 mg/kg to about 32 mg/kg, about 24 mg/kg to about 30 mg/kg, about 24 mg/kg to about 28 mg/kg, about 24 mg/kg to about 26 mg/kg, about 26 mg/kg to about 50 mg/kg, about 26 mg/kg to about 48 mg/kg, about 26 mg/kg to about 46 mg/kg, about 26 mg/kg to about 44 mg/kg, about 26 mg/kg to about 42 mg/kg, about 26 mg/kg to about 40 mg/kg, about 26 mg/kg to about 38 mg/kg, about 26 mg/kg to about 36 mg/kg, about 26 mg/kg to about 34 mg/kg, about 26 mg/kg to about 32 mg/kg, about 26 mg/kg to about 30 mg/kg, about 26 mg/kg to about 28 mg/kg, about 28 mg/kg to about 50 mg/kg, about 28 mg/kg to about 48 mg/kg, about 28 mg/kg to about 46 mg/kg, about 28 mg/kg to about 44 mg/kg, about 28 mg/kg to about 42 mg/kg, about 28 mg/kg to about 40 mg/kg, about 28 mg/kg to about 38 mg/kg, about 28 mg/kg to about 36 mg/kg, about 28 mg/ kg to about 34 mg/kg, about 28 mg/kg to about 32 mg, about 28 mg/kg to about 30 mg/kg, about 30 mg/kg to about 50 mg/kg about 30 mg/kg to about 48 mg/kg, about 30 mg/kg to about 46 mg/kg, about 30 mg/kg to about 44 mg/kg, about 30 mg/kg to about 42 mg/kg, about 30 mg/kg to about 40 mg/kg, about 30 mg/kg to about 38 mg/kg, about 30 mg/kg to about 36 mg/kg, about 30 mg/kg to about 34 mg/kg, about 30 mg/kg to about 32 mg/kg, about 32 mg/kg to about 50 mg/kg, about 32 mg/kg to about 48 mg/kg, about 32 mg/kg to about 46 mg/kg, about 32 mg/kg to about 44 mg/kg, about 32 mg/kg to about 42 mg/kg, about 32 mg/kg to about 40 mg/kg, about 32 mg/kg to about 38 mg/kg, about 32 mg/kg to about 36 mg/kg, about 32 mg/kg to about 34 mg/kg, about 34 mg/kg to about 50 mg/kg, about 34 mg/kg to about 48 mg/kg, about 34 mg/kg to about 46 mg/kg, about 34 mg/kg to about 44 mg/kg, about 34 mg/kg to about 42 mg/kg, about 34 mg/kg to about 40 mg/kg, about 34 mg/kg to about 38 mg/kg, about 34 mg/kg to about 36 mg/kg, about 36 mg/kg to about 50 mg/kg, about 36 mg/kg to about 48 mg/kg, about 36 mg/kg to about 46 mg/kg, about 36 mg/kg to about 44 mg/kg, about 36 mg/kg to about 42 mg/kg, about 36 mg/kg to about 40 mg/kg, about 36 mg/kg to about 38 mg/kg, about 38 mg/kg to about 50 mg/kg, about 38 mg/kg to about 48 mg/kg, about 38 mg/kg to about 46 mg/kg, about 38 mg/kg to about 44 mg/kg, about 38 mg/kg to about 42 mg/kg, about 38 mg/kg to about 40 mg/kg, about 40 mg/kg to about 50 mg/kg, about 40 mg/kg to about 48 mg/kg, about 40 mg/kg to about 46 mg/kg, about 40 mg/kg to about 44 mg/kg, about 40 mg/kg to about 42 mg/kg, about 42 mg/kg to about 50 mg/kg, about 42 mg/kg to about 48 mg c, about 42 mg/kg to about 46 mg/kg, about 42 mg/kg to about 44 mg/kg, about 44 mg/kg to about 50 mg/kg, about 44 mg/kg to about 48 mg/kg, about 44 mg/kg to about 46 rng/kg, about 46 mg/kg to about 50 mg/kg, about 46 mg/kg to about 48 mg/kg, or about 46 mg/kg to about 50 mg/kg)

In some embodiments, themethods described herein include administering to a subject about 10 mg/kg to about 400 mg/kg of body weight of sodium phen.ylbutyrate per day (e.g, about 10 mg/kg to about 380 mg/kg, about 10 mg/kg to about 360 mg/kg, about 10 mg/kg to about 340 mg/kg, about 10 mg/kg to about 320 mg/kg, about 10 mg/kg to about 300 mg/kg, about 10 mg/kg to about 280 mg/kg, about 10 mg/kg to about 260 mg/kg, about 10 mg/kg to about 240 mg/kg, about 10 mg/kg to about 220 mg/kg, about 10 mg/kg to about 200 mg/kg, about 10 mg/kg to about 180 mg/kg, about 10 mg/kg to about 160 mg/kg, about 10 mg/kg to about 140 mg/kg, about 10 mg/kg to about 120 mg/kg, about 10 mg/kg to about 100 mg/kg, about 10 mg/kg to about 80 mg/kg, about 10 mg/kg to about 60 mg/kg, about 10 mg/kg to about 40 mg/kg, about 10 mg/kg to about 20 mg/kg, about 20 mg/kg to about 400 mg/kg, about 20 mg/kg to about 380 mg/kg, about 20 mg/kg to about 360 mg/kg, about 20 mg/kg to about 340 mg/kg, about 20 mg/kg to about 320 mg/kg, about 20 mg/kg to about 300 mg/kg, about 20 mg/kg to about 280 mg/kg, about 20 mg/kg to about 260 mg/kg, about 20 mg/kg to about 240 mg/kg, about 20 mg/kg to about 220 mg/kg, about 20 mg/kg to about 200 mg/kg, about 20 mg/kg to about 180 mg/kg, about 20 mg/kg to about 160 mg/kg, about 20 mg/kg to about 140 mg/kg, about 20 mg/kg to about 120 mg/kg, about 20 mg/kg to about 100 mg/kg, about 20 mg/kg to about 80 mg/kg, about 20 mg/kg to about 60 mg/kg, about 20 mg/kg to about 40 mg/kg, about 40 mg/kg to about 400 mg/kg, about 40 mg/kg to about 380 mg/kg, about 40 mg/kg to about 360 mg/kg, about 40 mg/kg to about 340 mg/kg, about 40 mg/kg to about 320 mg/kg, about 40 mg/kg to about 300 mg/kg, about 40 mg/kg to about 280 mg/kg, about 40 mg/kg to about 260 mg/kg, about 40 mg/kg to about 240 mg/kg, about 40 mg/kg to about 220 mg/kg about 40 mg/kg to about 200 mg about 40 mg/kg to about 180 mg/kg, about 40 mg/kg to about 160 mg/kg, about 40 mg/kg to about 140 mg/kg, about 40 mg/kg to about 120 mg/kg, about 40 mg/kg to about 100 mg/kg, about 40 mg/kg to about 80 mg/kg, about 40 mg/kg to about 60 mg/kg, about 60 mg/kg to about 400 mg/kg, about 60 mg/kg to about 380 mg/kg, about 60 mg/kg to about 360 mg/kg, about 60 mg/kg to about 340 mg/kg, about 60 mg/kg to about 320 mg/kg, about 60 mg/kg to about 300 mg/kg, about 60 mg/kg to about 280 mg/kg, about 60 mg/kg to about 260 mg/kg, about 60 mg/kg to about 240 mg/kg., about 60 mg/kg to about 220 mg/kg, about 60 mg/kg to about 200 mg/kg, about 60 mg/kg to about 180 mg/kg, about 60 mg/kg to about 160 mg/kg, about 60 mg/kg to about 140 mg/kg, about 60 mg/kg to about 120 mg/kg, about 60 mg/kg to about 100 mg/kg, about 60 mg/kg to about 80 mg/kg, about 80 mg/kg to about 400 mg/kg, about 80 mg/kg to about 380 mg/kg, about 80 mg/kg to about 360 mg/kg, about 80 mg/kg to about 340 mg/kg, about 80 mg/kg to about 320 mg/kg, about 80 mg/kg to about 300 mg/kg, about 80 mg/kg to about 280 mg/kg, about 80 mg/kg to about 260 mg/kg, about 80 mg/kg to about 240 mg/kg, about 80 mg/kg to about 220 mg/kg, about 80 mg/kg to about 200 mg/kg, about 80 mg/kg to about 180 mg/kg, about 80 mg/kg to about 160 mg/kg, about 80 mg/kg to about 140 mg/kg, about 80 mg/kg to about 120 mg/kg, about 80 mg/kg to about 100 mg/kg, about 100 mg/kg to about 400 mg/kg, about 100 mg/kg to about 380 mg/kg, about 100 mg/kg to about 360 mg/kg, about 100 mg/kg to about 340 mg/kg, about 100 mg/kg to about 320 mg/kg, about 100 mg/kgto about 300 mg/kg, about 100 mg/kg to about 280 mg/kg, about 100 mg/kg to about 260 mg/kg, about 100 mg/kg to about 240 mg/kg, about 100 mg/kg to about 220 mg/kg, about 100 mg/kg to about 200 mg/kg, about 100 mg/kg to about 180 mg/kg, about 100 mg/kg to about 160 mg/kg, about 100 mg/kg to about 140 mg/kg, about 100 mg/kg to about 12.0 mg/kg, about 120 mg/kg to about 400 mg/kg, about 120 mg/kg to about 380 mg/kg, about 120 mg/kg to about 360 mg/kg, about 120 mg/kg to about 340 mg/kg, about 120 mg/kg to about 320 mg/kg, about 120 mg/kg to about 300 mg/kg, about 120 mg/kg to about 280 mg/kg, about 120 mg/kg to about 260 mg/kg, about 120 mg/kg to about 240 mg/kg, about 120 mg/kg to about 220 mg/kg, about 120 mg/kg to about 200 mg/kg, about 120 mg/kg to about 180 mg/kg, about 120 mg/kg to about 160 mg/kg, about 120 mg/kg to about 140 mg/kg, about 140 mg/kg to about 400 mg/kg, about 140 mg/kg to about 380 mg/kg, about 140 mg/kg to about 360 mg/kg, about 140 mg/kg to about 340 mg/kg, about 140 mg/kg to about 320 mg/kg, about 140 mg/kg to about 300 mg/kg, about 140 mg/kg to about 280 mg/kg, about 140 mg/kg to about 260 mg/kg, about 140 mg/kg to about 240 mg/kg, about 140 mg/kg to about 220 mg/kg, about 140 mg/kg to about 200 mg/kg, about 140 mg/kg to about 180 mg/kg, about 140 mg/kg to about 160 mg/kg, about 160 mg/kg to about 400 mg/kg, about 160 mg/kg to about 380 mg/kg, about 160 mg/kg to about 360 mg/kg, about 160 mg/kg to about 340 mg/kg, about 160 mg/kg to about 320 mg/kg, about 160 mg/kg to about 300 mg/kg, about 160 mg/kg to about 280 mg/kg, about 160 mg/kg to about 260 mg/kg, about 160 mg/kg to about 240 mg/kg, about 160 mg/kg to about 220 mg/kg, about 160 mg/kg to about 200 mg/kg, about 160 mg/kg to about 180 mg/kg, about 180 mg/kg to about 400 mg/kg, about 180 mg/kg to about 380 mg/kg, about 180 mg/kg to about 360 mg/kg, about 180 mg/kg to about 340 mg/kg, about 180 mg/kg to about 320 mg/kg, about 180 mg/kg to about 300 mg/kg, about 180 mg/kg to about 280 mg/kg, about 180 mg/kg to about 260 mg/kg, about 180 mg/kg to about 240 mg/kg, about 180 mg/kg to about 220 mg/kg, about 180 mg/kg to about 200 mg/kg, about 200 mg/kg to about 400 mg/kg, about 200 mg/kg to about 380 mg/kg, about 200 mg/kg to about 360 mg/kg, about 200 mg/kg to about 340 mg/kg, about 200 mg/kg to about 32.0 mg/kg, about 200 mg/kg to about 300 mg/kg, about 200 mg/kg to about 280 mg/kg, about 200 mg/kg to about 260 mg/kg, about 200 mg/kg to about 240 mg/kg, about 200 mg/kg to about 220 mg/kg, about 220 mg/kg to about 400 mg/kg, about 220 mg/kg to about 380 mg/kg, about 220 mg/kg to about 360 mg/kg, about 220 mg/kg to about 340 mg/kg, about 220 mg/kg to about 320 mg/kg, about 220 mg/kg to about 300 mg/kg, about 220 mg/kg to about 280 mg/kg, about 220 mg/kg to about 260 mg/kg, about 220 mg/kg to about 240 mg/kg, about 240 mg/kg to about 400 mg/kg, about 240 mg/kg to about 380 mg/kg, about 240 mg/kg to about 360 mg/kg, about 240 mg/kg to about 340 mg/kg, about 240 mg/kg to about 320 mg/kg, about 240 mg/kg to about 300 mg/kg, about 240 mg/kg to about 280 mg/kg, about 240 mg/kg to about 260 mg/kg, about 260 mg/kg to about 400 mg/kg, about 260 mg/kg to about 380 mg/kg, about 260 mg/kg to about 360 mg/kg, about 260 mg/kg to about 340 mg/kg, about 260 mg/kg to about 320 mg/kg, about 260 mg/kg to about 300 mg/kg, about 260 mg/kg to about 280 mg/kg, about 280 mg/kg to about 400 mg/kg, about 280 mg/kg to about 380 mg/kg, about 280 mg/kg to about 360 mg/kg, about 280 mg/kg to about 340 mg/kg, about 280 mg/kg to about 320 mg/kg, about 280 mg/kg to about 300 mg/kg, about 300 mg/kg to about 400 mg/kg, about 300 mg/kg to about 380 mg/kg, about 300 mg/kg to about 360 mg/kg, about 300 mg/kg to about 340 mg/kg, about 300 mg/kg to about 320 mg/kg, about 320 mg/kg to about 400 mg/kg, about 320 mg/kg to about 380 mg/kg, about 320 mg/kg to about 360 mg/kg, about 320 mg/kg to about 340 mg/kg, about 340 mg/kg to about 400 mg/kg, about 340 mg/kg to about 380 mg/kg, about 340 mg/kg to about 360 mg/kg, about 360 mg/kg to about 400 mg/kg, about 360 mg/kg to about 380 mg/kg, or about 380 mg/kg to about 400 mg/kg)

In some embodiments, TURSO is administered in an amount of about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, or about 70 mg/kg of body weight per day. In some embodiments, sodium phen.ylbutyrate is administered in an amount of about 10 mg/kg, about 2.0 mg/kg, about 30 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, about 320 mg/kg, about 340 mg/kg, about 360 mg/kg, about 380 mg/kg, or about 400 mg/kg of body weight per day.

The methods desctibed herein can be used for treating or ameliorating at least one symptom of ALS in a subject, slowing ALS disease progression, increasing survival time of a subject having one or more symptoms of ALS, preventing or reducing at least one adverse events (e.g., serious adverse events) associated with ALS or its treatment, and reducing the deterioration of, maintaining or improving muscle strength, respiratory muscle/pulmonary function and/or fine motor skin. The methods can also be used for prophylactically treating a subject at risk for developing ALS (e.g., a subject with a family history of ALS) or suspected to be developing ALS (e.g., a subject displaying at least one symptom of ALS, a symptom of upper motor neuron degeneration, and/or a symptom of lower motor neuron degeneration, but not enough symptoms at that time to support a full diagnosis of ALS). The methods are useful for ameliorating at least one symptom of lower motor neuron degeneration or upper motor neuron degeneration.

The methods disclosed herein are also useful for preventing or reducing constipation (e.g., constipation associated with ALS), and ameliorating at least one symptom of benign fasciculation syndrome or cramp fasciculation syndrome.

As disclosed herein, the methods can be used for treating a subject diagnosed with ALS, at risk for developing ALS, or suspected as having ALS. The subject may, for example, have been diagnosed with ALS for 24 months or less (e.g., any of the subranges within this range described herein). For example, the subject may have been diagnosed with ALS for 1 week or less, or on the same day that the presently disclosed treatments are administered. The subject may have shown one or more symptoms of ALS for 24 months or less (e.g., any of the subranges within this range described herein), have an ALS disease progression rate (ΔFS) of about 0.50 or greater (e.g., any of the subranges within this range described herein), have an ALSFRS-R score of 40 or less (e.g., any of the subranges within this range described herein), have lost on average about 0.8 to about 2 ALSFRS-R points per month (e.g. any of the subranges within this range described herein) over the previous 3-12 months, have a mutation in one or more genes selected from the group consisting of: SOD1, C₉ORF72, ANG, TARDBP, VCP, VAPB, SQSTM1, DCTN1, FUS, UNC₁₃A, ATXN2, HNRNPA1, CHCHD10, MOBP, C₂₁ORF2, NEK1, TUB A4A, TBK1, MATR₃, PFN1, UBQLN2, TAF15, OPTN, and TDP-43, and/or have a CSF or blood level of pNF-H of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher). In some embodiments, the serum pNF-H level of subjects in the methods described herein can be about 70 to about 1200 pg/mL (e.g., about 70 to about 1000, about 70 to about 800, about 80 to about 600, or about 90 to about 400 pg/mL). In some embodiments, the CSF pNF-H levels of subjects in the methods described herein can be about 1000 to about 5000 pg/mL (e.g., about 1500 to about 4000, or about 2000 to about 3000 pg; /mL). The subject may have a CSF or blood level of NfL of about 50 pg/mL or higher (e.g., about 60, 70, 80, 90, 100, 110, 120, 130, 140, 150, 160, 170, 180, 190, 200, 210, 220, 230, 240, or 250 pg/mL or higher). In some embodiments, the serum NfL level of subjects in the methods described herein can be about 50 to about 300 pg/mL (e.g., any of the subranges within this range described herein). In some embodiments, the CSF NFL level of subjects in the methods described herein can be about 2000 to about 40,000 pg/mL (e.g., any of the subranges within this range described herein)

Methods described in the present disclosure can include treatment of ALS per se, as wel as treatment for one or more symptoms of ALS. “Treating” ALS does not require 100% abolition of the disease or disease symptoms in the subject. Any relief or reduction in the severity of symptoms or features of the disease is contemplated. “Treating” ALS also refers to a delay in onset of symptoms (e.g., in prophylaxis treatment) or delay in progression of symptoms or the loss of function associated with the disease. “Treating” ALS also refers to eliminating or reducing one or more side effects of a treatment (e.g, those caused by any of the therapeutic agents for treating ALS disclosed herein or known in the art). “Treating” ALS also refers to eliminating or reducing one or more direct or indirect effects of ALS disease progression, such as an increase in the number of falls, lacerations, or GI issues. The subject may not exhibit signs of ALS but may be at risk for ALS. For instance, the subject may carry mutations in genes associated with ALS, have family history of having ALS, or have elevated biomarker levels suggesting a risk of developing ALS. The subject may exhibit early signs of the disease or display symptoms of established or progressive disease. The disclosure contemplates any degree of delay in the onset of symptoms, alleviation of one or more symptoms of the disease, or delay in the progression of any one or more disease symptoms (e.g., any improvement as measured by ALSFRS-R, or maintenance of an ALSFRS-R rating (signaling delayed disease progression)). Any relief or reduction in the severity of symptoms or features of benign fasciculation syndrome and cramp-fasciculation syndrome are also contemplated herein.

The treatment provided in the present disclosure can be initiated at any stage during disease progression. For example, treatment can be initiated prior to onset (e.g., for subjects at risk for developing ALS), at symptom onset or immediately following detection of ALS symptoms, upon observation of any one or more symptoms (e.g., muscle weakness, muscle fasciculations, and/or muscle cramping) that would lead a skilled practitioner to suspect that the subject may be developing ALS. Treatment can also be initiated at later stages. For example, treatment may be initiated at progressive stages of the disease, e.g., when muscle weakness and atrophy spread to different parts of the body and the subject has increasing problems with moving. At or prior to treatment initiation, the subject may suffer from tight and stiff muscles (spasticity), from exaggerated reflexes (hyperreflexia), from muscle weakness and atrophy, from muscle cramps, and/or from fleeting twitches of muscles that can be seen under the skin (fasciculations), difficulty swallowing (dysphagi a), speaking or forming words (dysarthria).

Treatment methods can include a single administration, multiple administrations, and repeating administration as required for the prophylaxis or treatment of ALS, or at least one symptom of ALS. The duration of prophylaxis treatment can be a single dosage or the treatment may continue (e.g., multiple dosages), e.g., for years or indefinitely for the lifespan of the subject. For example, a subject at risk for ALS may be treated with the methods provided herein for days, weeks, months, or even years so as to prevent the disease from occurring or fulminating. In some embodiments treatment methods can include assessing a level of disease in the subject prior to treatment, during treatment, and/or after treatment. The treatment provided herein can be administered one or more times daily, or it can be administered weekly or monthly. In some embodiments, treatment can continue until a decrease in the level of disease in the subject is detected. The methods provided herein may in some embodiments begin to show efficacy (e.g., alleviating one or more symptoms of ALS, improvement as measured by the ALSMS-R, or maintenance of an ALSFRS-R rating) less than 60 days (e.g., less than 50, 45, 40, 35, 30, 25, 20, 15, or 10 days) after the initial administration, or after less than 60 administrations (e.g., less than 50, 45, 40, 35, 30, 25, 20, 15, or 10 administrations).

The terms “administer”, “administering”, or “administration” as used herein refers to administering drugs described herein to a subject using any art-known method, e.g., ingesting, injecting, implanting, absorbing, or inhaling, the drug, regardless of form. In some embodiments, one or more of the compounds disclosed herein can be administered to a subject by ingestion orally and/or topically (e.g., nasally). For example, the methods herein include administration of an effective amount of compound or compound composition to achieve the desired or stated effect. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Following administration of the bile acid or a pharmaceutically acceptable salt thereof and the phenylbutyrate compound, the subject can be evaluated to detect ; assess, or determine their level of ALS disease. In some embodiments, treatment can continue until a change (e.g., reduction) in the level of disease in the subject is detected.

Upon improvement of a patient's condition (e.g., a change e.g., decrease) in the level of disease in the subject), a maintenance dose of a compound, composition or combination of this disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

IV. Symptom and Outcome Measurements

This disclosure further provides methods of evaluating ALS symptoms, monitoring ALS progression and evaluating the subject's response to the treatment methods. Non-limiting examples include physical evaluation by a physician, weight, Electrocardiogram (ECG), ALS Functional Rating Scale (ALSFRS or ALSFRS-R) score, respiratory function, muscle strength, cognitive/behavioral function, quality of life, and speech analysis.

Respiratory function of the subject can be measured by e.g. vital capacity (including forced vital capacity and slow vital capacity), maximum mid-expiratory flow rate (MMERF), forced vital capacity (FVC), and forced expiratory volume in 1 second (FEV₁). Muscle strength can be evaluated by e.g. hand held dynamometry (HHD), hand grip strength dynamometry, manual muscle testing (NtMT), electrical impedance myography (ELM), Maximum Voluntary Isometric Contraction Testing (MVICT), motor unit number estimation (MUNE), Accurate Test of Limb Isometric Strength (ATLIS), or a combination thereof. Cognitive/behavior function can be evaluated by e.g. the ALS Depression Inventory (ADI-12), the Beck Depression Inventory (BDI), and the Hospital Anxiety Depression Scale (HADS) questionnaires. Quality of life can be evaluated by e.g. the ALS Assessment Questionnaire (ALSAQ-40). The Akt level, Akt phosphorylation and/or pAktdAkt ratio can also be used to evaluate a subject's disease progression and response to treatment (See e.g., WO2012/160563).

The levels of biomarkers in the subject's CSF or blood samples are useful indicators of the subject's ALS progression and responsiveness to the methods of treatment provided herein. Biomarkers such as but not limited to, phosphorylated neurofilament heavy chain (pNT-H), neurofilament medium chain, neurofilament light chain (NFL), S100-β, cystatin C, chitotriosidase, C-reactive protein (CRP), TDP-43, uric acid, and certain micro RNAs, can be analyzed for this purpose. Urinalysis can also be used for assessing the subject's response to treatment. Levels of biomarkers such as but not limited to p75ECD and ketones in the urine sample can be analyzed. Levels of creatinine can be measured in the urine and blood samples. In some embodiments, the methods provided herein result in increased or decreased ketone levels in the subject's urine sample. Medical imaging, including but not limited to MR(and PET imaging of markers such as Translocator protein (TSPO), may also be utilized.

In some embodiments, YKL-40 alone or in correlation with the ALSFRS-R score may be used as indicators of the subject's ALS progression and responsiveness to the methods of treatment provided herein. For example, administration with the compositions described herein may result in at least about a 0.5 ng/mL reduction (e.g., about a 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 nglinL, 14 ng/mL, 15 ng/mL, 16 ng/mL, 17 ng/mL, 18 ng/mL, 19 ng/mL, 20 ng/mL, or any subranges within this range, reduction) in the level of plasma YKL-40 as compared to a pre-administration level or to a subject that has not been treated with the compositions described herein. In some cases, after administration with any of the compositions described herein (e.g., a composition comprising TURSO and sodium phenylbutyrate) for a period of time (e.g., about 12 weeks to about 24 weeks), the subjects may have a plasma level of YKL-40 of about 25 ng/mL to about 38 ng/mL (e.g., about 26, 27, 28, 29, 30, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32, 33, 34, 35, 36, or 37 ng/mL).

In one aspect, provided herein are methods of treating at least one symptom of ALS in a human subject, the method comprising: (a) administering to the human subject TURSO and sodium phenylbutyrate (e.g., administering a composition comprising Taurursodiol (TURSO) and sodium phenylbutyrate), (b) determining or having determined that the level of YKL-40 in a biological sample of the subject is higher than about 33 ng/mL (e.g. higher than about 34, 35, 36, 37, or 38 ng/mL), and (c) continuing to administer to the subject TURSO and sodium phenylbutyrate.

In one aspect, provided herein are methods of treating at least one symptom of ALS in a human subject, the method comprising: (a) determining or having determined a first level of YKL-40 in a first biological sample of a human subject, (b) administering to the human subject TURSO and sodium phenylbutyrate (e.g. administering a composition comprising TURSO and sodium phenylbutyrate), (c) determining or having determined a second level of YKL-40 in a second biological sample of a human subject, and (d) continuing administering to the subject TURSO and sodium phenylbutyrate if the second level of YKL-40 is higher than the first level of YKL-40.

In another aspect, provided herein are methods of treating at least one symptom of ALS in a human subject, the method comprising: (a) determining or having determined a first level of YKL-40 in a first biological sample of a human subject, (b) administering to the human subject TURSO and sodium phenylbutyrate (e.g., administering a composition comprising TURSO and sodium phen.ylbutyrate), (c) determining or having determined a second level of YKL-40 in a second biological sample of a human subject, and (d) continuing administering to the subject TURSO and sodium phenylbutyrate if the second level of YKL-40 is lower than the first level. In some embodiments, step (b) comprises administering to the human subject TURSO and sodium phenylbutyrate for about 24 weeks and step (d) comprises continuing administering to the human subject TURSO and sodium phenylbutyrate if the second level of YIKL-40 is lower than the first level of YKL-40 by about 0.75 ng/mL or less (e.g., about 0.5 ng/mL or less). In some embodiments, the second level of YKL-40 is lower than the first level of YKL-40 by about 0.70 ng/mL, 0.65 ng/mL, 0.60 ng/mL, 0.550 ng/mL, 0.50 ng/mL, 0.45 ng/mL, 0.40 ng,/mL, 0.35ng/mL, 0.30 ng/mL, 0.25 ng/mL, 0.20 ng/mL, 0.15 ng/mL, or 0.10 ng/mL. In some embodiments, step (b) comprises administering to the human subject TURSO and sodium phenylbutyrate for about 24 weeks and step (d) comprises continuing administering to the human subject TURSO and sodium phenylbutyrate if the second level of YKL-40 is lower than the first level of VKL-40 by about 0.75 ng/mL or more example, the second level of YKL-40 can be lower than the first level of YKL-40 by about 0.80 ng/mL, 0.90 ng/mL, 1.0 ng/mL or more.

In some embodiments, CRP levels alone or in correlation with the ALSFRS-R score may be used as indicators of the subject's ALS progression and responsiveness to the methods of treatment provided herein. For example, administration with the compositions described herein may result in about a 50%, 45%, 40%, 35%, 30%, 25%, 20% or any subranges within this range, reduction in the level of plasma. CRP as compared to a subject that has not been treated with the compositions described herein. In some cases, after administration with any of the compounds described herein (e.g., a combination of TURSO and sodium phenylbutyrate) for a period of time (e.g., about 12 weeks to about 24 weeks), the subjects may have a plasma level of CRP of about 1000 ng/mL to about 2250 ng/mL (e.g., about 1050, 1100, 1150, 1200, 1250, 1300, 1350, 1400, 1450, 1500, 1550, 1600, 1650, 1700, 1750, 1800, 1900, 1950, 2000, 2050, 2100, 2150, or 2200 ng/mL).

Also provided herein are methods of treating at least one symptom of ALS in a human subject, the methods comprising: (a) administering to the human subject TURSO and sodium phenylbutyrate, (b) determining or having determined that the level of C-reactive protein (CRP) in a biological sample of the subject is higher than about 1835 ng/mL, and (c) continuing to administer to the subject TURSO and sodium phenylbutyrate. For example, step (b) can include determining or having determined that the level of CRP in the biological sample is higher than about 1850 ng/'mL, 1900 ng/mL, 2000 ng,/mL, 2200 ng/'mL, 2400 ng/mL, 2600 ng/mL, or 2800 ng/mL or more.

In one aspect, provided herein are methods of treating at least one symptom of ALS in a human subject, the methods comprising: (a) determining or having determined a first level of C-reactive protein (CRP) in a first biological sample of a human subject, (b) administering to the human subject TURSO and sodium phenylbutyrate, (c) determining or having determined a second level of CRP in a second biological sample of the human subject, and (d) continuing administering to the subject TURSO and sodium phenylbutyrate if the second level of CRP is higher than the first level of CRP.

In another aspect, provided herein are methods of treating at least one symptom of FLS in a human subject, the methods comprising (a) determining or having determined a first level of C-reactive protein (CRP) in a first biological sample of a human subject, (b) administering to the human subject TURSO and sodium phenylbutyrate, (c) determining or having determined a second level of CRP in a second biological sample of the human subject, and (d) continuing administering to the subject TURSO and sodium phenvlbutyrate if the second level of CRP is lower than the first level of CRP.

In some embodiments of any of the above aspects, the methods can include administering about 1 gram of TURSO and about 3 grams of sodium phenylbutyrate once a day or twice a day, orally or through a feeding tube. For example, the methods can include administering about 1 gram of TURSO once a day and about 3 grams of sodium phenylbutyrate once a day for about 14 days, followed by administering about 1 gram of TURSO twice a day and about 3 grams of sodium phenylbutyrate twice a day.

### Mitochondria Dysfunction

Mitochondrial dysfunction is widespread in neurodegenerative disease. In Alzheimer's disease, the mitochondrial membrane potential of cells is markedly reduced, glucose metabolism by the mitochondria is impaired, and the permeability of the mitochondria is increased. Mitochondria have been observed to mediate multiple apoptotic pathways resulting in neuronal death in Alzheimer's disease.

PINK1 and Parkin are both mitochondrial quality control proteins. Mutations or lack of these proteins is strongly linked to Parkinson's disease, MPTP, a molecule used to induce permanent symptoms of Parkinson's, acts through the disruption of complex I of the mitochondria, causing mitochondrial dysfunction, alteration of the redox state of the cell, and apoptosis.

It has been directly shown in cell culture that the mutant Huntingtin gene and its resultant protein, thought to be the primary mediator of Huntington's disease, results in a loss of membrane potential and decreased expression of critical oxidative phosphorylati on genes in the mitochondria. Huntington's disease pathology has also been linked to a decrease in the number of mitochondria present in the central nervous system.

Mitochondrial dyslocalization, energy metabolism impairment, and apoptotic pathways are thought to mediate Amyotrophic lateral sclerosis. Mitochondria from affected tissues have also been shown to overproduce reactive oxygen metabolites and leak them to the cytosol.

In many neurodegenerative diseases, mitochondria overproduce free radicals, cause a reduction in energy metabolism, have increased permeability, have decreased membrane potential, have decreased antioxidants, leak metal ions into the cell, alter the redox state of the cell, and lead the cell down pro-apoptotic pathways. A need therefore exists for agents that can alter and reduce mitochondrial dysfunction mechanisms.

Also included are methods of reducing mitochondrial dysfunction, or treating at least one symptom associated with mitochondrial dysfunction, preventing the time of onset of, or slowing the development of a disease or condition related to mitochondrial dysfunction.

### Muscle Strength

The muscle strength of a subject can be evaluated using known methods in the art. Quantitative strength measures generally demonstrate a linear, predictable strength loss within an ALS patient. Tufts Quantitative Neuromuscular Examination (TQNE) can be used to provide quantitative measurements using a fixed strain gauge. TQNE measures isometric strength of 20 muscle groups and produces interval strength data in both strong and weak muscles (See e.g., Andres et al., Neurology 36:937-941, 1986). Hand-held dynamometry (HHD) tests isometric strength of specific muscles in the arms and legs and produces interval level data (See e.g., Shefne J M Neurotherapeutics 14:154-160, 2017).

Accurate Test of Limb Isometric Strength (ATLIS) can be used to measure both strong and weak muscle groups using a fixed, wireless load cell (See e.g., Andres et al., Muscle Nerve 56(4):710-715, 2017). Force in twelve muscle groups are evaluated in an ATLIS testing, which reflect the subject's strength in the lower limbs, upper limbs, as well as the subject's grip strength. In some embodiments, ATLIS testing detects changes in muscle strength before any change in function is observed.

The methods provided herein may improve, maintain, or slow down the deterioration of a subject's muscle strength (e.g., lower limb strength, upper limb strength, or grip strength), as evaluated by any suitable methods described herein. The methods may result in improvement of the subject's upper limb strength more significantly than other muscle groups. For example, the effect on muscle strength can be reflected in one or more muscle groups selected from quadriceps, biceps, hamstrings, triceps, and anterior tibialis.

Muscle strength can be assessed by HHD, hand grip strength dynamometry, MMT, EIM, MVICT, MUNE, ATLIS, or a combination thereof, before, during and/or after the administration of a bile acid or a pharmaceutically acceptable salt thereof and a phenylbutyrate compound.

In some embodiments, the muscle strength is assessed by ATLIS. The total ATLIS score as well as the upper extremity and lower extremity ATLIS scores can be assessed. The methods of the present disclosure can result in a rate of decline in the total ATLIS score of a subject of about 3.50 PPN/month or less (e.g., about 3.45, 3.40, 3.35, 3.30, 3.25, 3.20, 3.15, 3.10, 3.05, 3.00 PPN/month or less). The methods of the present disclosure can also results in a reduction of the mean rate of decline in the total ATLIS score of a subject by at least about 0.2 PPN/month (e.g., at least about 0.25, 0.30, 0.35, 0.40, 0.45, or 0.50 PPN/month) as compared to a control subject not receiving the administration. The mean rate of decline in the upper extremity ATLIS score of a subject can be reduced by at least about 0.50 PPN/month (e.g., at least about 0.55, 0.60, 0.65, 0.70, 0.75, 0.80, 0.85, or 0.90 PPN/month) as compared to a control subject not receiving the administration described herein. The mean rate of decline in the lower extremity ATLIS score of a subject can be reduced by at least about 0.20 PPN/month (e.g., at least about 0.25, 0.30, 0.3:5, 0.40, 0.45, 0.50, 0.55, or 0.60 PPN/month) as compared to a control subject not receiving the administration described herein. In some embodiments, improvement or maintenance of the subject's muscle strength may begin to occur less than 60 days (e.g., less than 55, 50, 45, 40, 30, 25, or 20 days) following the initial administration. PPN represents the percentage of predicted normal strength based on age, sex weight and height.

### Pulmonary Funcion

ALS is a progressive neurodegenerative disease that ultimately leads to respiratory failure and death. Pulmonary function tests, such as vital capacity (VC), maximum mid-expiratory flow rate (MMERF), forced vital capacity (FVC), slow vital capacity (SVC), and forced expiratory volume in 1 second (FEV₁), can be used to monitor ALS progression and/or the subject's response to treatment. On average, the rate of respiratory function decline of an ALS patient measured by Vital Capacity (VC) can be about 2.24% of predicted (±6.9) per month. In some embodiments, measures from pulmonary function tests are associated with survival (See e.g., Moufavi et al. Iran J Neurol 13(3): 131-137, 2014). Additional measures, such as maximal inspiratory and expiratory pressures, arterial blood gas measurements, and overnight oximetry, may provide earlier evidence of dysfunction. Comparison of vital capacity in the upright and supine positions may also provide an earlier indication of weakening ventilatory muscle strength.

The methods provided herein may improve or maintain the subject's respiratory muscle and/or pulmonary function, or slow down the deterioration of the subject's respiratory muscle and/or pulmonary function. A subject's respiratory muscle and/or pulmonary function can be evaluated by any of the suitable methods described herein or otherwise known in the art. In some embodiments, the respiratory muscle function of a human subject is assessed based on the subject's SVC. In some embodiments of any of the methods of improving, maintaining, or slowing down the deterioration of respiratory muscle function in a human subject described herein, the treatment results in a mean rate of decline in the SVC of the subject of about 3.50 PPN/month or less (e.g., about 3.45, 3.40, 3.35. 3.30, 3.25, 3.20, 3.15, 3.10, 3.05, or 3.00 PPN/month or less). In some embodiments, the treatment reduces the mean rate of decline in the SVC of the subject by at least about 0.5 PPN/month (e.g., at least about 0.55, 0.60, 0.65, 0.70, 0.75, 0,80, 0.85, 0,90, 0.95, or 1.00 PPN/month) as compared to a control subject not receiving the treatment. In some embodiments, improvement or maintenance of the subject's pulmonary function may begin to occur less than 60 days (e.g., less than 55, 50, 45, 40, 30, 25, or 20 days) following the initial administration. In some embodiments, the subject's pulmonary function progresses less than expected after fewer than 60 days following the initial administration.

### Adverse Events

Subjects treated with any of the methods provided herein may present fewer adverse events (e.g., any of the adverse events disclosed herein), or present one or more of the adverse events to a lesser degree than control subjects not receiving the treatment. Exemplary adverse events include gastrointestinal related adverse events (e.g., abdominal pain, gastritis, nausea and vomiting, constipation, rectal bleeding, peptic ulcer disease, and pancreatitis); hematologic adverse events (e.g., aplastic anemia and ecchymosis); cardiovascular adverse events (e.g., arrhythmia and edema); renal adverse events (e.g., renal tubular acidosis); psychiatric adverse events (e.g., depression); skin adverse events (e.g., rash); and miscellaneous adverse events (e.g., syncope and weight gain). In some embodiments, the methods provided herein do not result in, or result in minimal symptoms of, constipation, neck pain, headache, falling, dry mouth, muscular weakness, falls, laceration, and Alanine Aminotransferase (ALT) increase. In some embodiments, the adverse events are serious adverse events, such as but not limited to respiratory adverse events, falls, or lacerations.

In some embodiments, administration of the combination of a bile acid and a phenylbutyrate compound can result in fewer adverse events (e.g., any of the adverse events disclosed herein), or less severe adverse events compared to administration of the bile acid or the phenylbutyrate compound alone.

The average survival time for an ALS patient may vary. The median survival time can be about 30 to about 32 months from symptom onset, or about 14 to about 20 months from diagnosis. The survival time of subjects with bulbar-onset ALS can be about 6 months to about 84 months from symptom onset, with a median of about 27 months. The methods provided herein may in some embodiments increase survival for a subject having ALS by at least one month (e.g., at least 2, 3, 4, 5, 6, 7, 8, 9, 10, 11, 12, 13, 14, 15, 16, 17, 18, 19, 20, 21, 22, 23, 24, 28, 32, 36, 40, 50, 60, 70, 80, or 90 months). Methods provided herein may in some embodiments delay the onset of ventilator-dependency or tracheostomy by at least one month (e.g., at least 2, 3, 4, 5, 6, 7, 8, 9, 10, 11, 12, 13, 14, 15, 16, 17, 18, 19, 20, 21, 22, 23, 24, 28, 32, 36, 40, 50, 60, 70, 80, or 90 months).

Methods provided herein may reduce disease progression rate wherein the average ALSFRS-R points lost per month by the subject is reduced by at least about 0.2 (e.g., at least about 0.25, 0.30, 0.35, 0.40, 0.45, 0.50, 0.55, 0.60, 0.65, 0.70, 0.75, 0.80, 0.85, 0.90, 0.95, 1.0, 1.05, 1.1, 1.15, 1.2, 1.25, 1.3, 1.35, 1.4, 1.45 or 1.5) as compared to a control subject not receiving the treatment. The methods provided herein may slow down the progression in one or more categories evaluated by the ALSFRS scale, including: speech, salivation, swallowing, handwriting, Cutting Food and Handling Utensils, Dressing and Hygiene, Turning in Bed and Adjusting Bed Clothes, Walking, Climbing Stairs, Dyspnea, Orthopnea, Respiratory insufficiency. In some embodiments, the methods provided herein improve or slow down deterioration of a subject's fine motor function, as evaluated by one or more categories of the ALSFRS-R scale (e.g., handwriting, cutting food and handling utensils, or dressing and hygiene).

.1n some embodiments, the methods provided herein are more effective in treating subjects that are about 18 to about 50 years old (e.g., about 18 to about 45, about 18 to about 40, about 18 to about 35, about 18 to about 30, about 18 to about 25, or about 18 to about 22 years old), as compared to subjects 50 years or older (e.g., 55, 60, 65, 70, 75, or 80 years or older). In some embodiments, the methods provided herein are more effective in treating subjects who have been diagnosed with ALS and/or who showed ALS symptom onset less than about 24 months (e.g., less than about 22, 20, 18, 16, 14, 12, 10, 8, 6, 4, 2, or 1 month), as compared to subjects who has been diagnosed with ALS and/or who showed ALS symptom onset more than about 24 months (e.g., more than about 26, 28, 30, 32, 34, 36, 40, 45, 50, 55, or 60 months). In some embodiments, the methods provided herein are more effective in treating subjects who have been diagnosed with ALS and/or who showed ALS symptom onset more than about 24 months (e.g., more than about 26, 28, 30, 32, 34, 36, 40, 45, 50, 55, or 60 months), as compared to subjects who has been diagnosed with ALS and/or who showed ALS symptom less than about 24 months (e.g., less than about 22, 20, 18, 16, 14, L, 10, 8, 6, 4, 2, or 1 month).

In some embodiments, responsiveness to the methods of treatment provided herein are gender-dependent. The methods provided herein can be more or less effective in treating female subjects as compared to male subjects. For instance, female subjects may show improvements (e.g., as measured by the ALSFRS-R or any other outcome measures described herein) earlier or later than male subjects when treated at similar stages of disease progression. Female subjects may in sonic embodiments show bigger or smaller improvements (e.g., as measured by the ALSFRS-R or any other outcome measures described herein) than male subjects when treated at similar stages of disease progression. The pharmacokinetics of the bile acid and the phenylbutyrate compound may be the same or different in female and male subjects.

### V. Additional Therapeutic Agents

The methods described herein can further include administering to the subject one or more additional therapeutic agents, e.g. in amounts effective for treating or achieving a modulation of at least one symptom of ALS. Any known ALS therapeutic agents known in the art can be used as an additional therapeutic agent. Exemplary therapeutic agents include riluzole (C₈H₅F₃N₂OS, e.g. sold under the trade names Rilutek® and Tiglutik®), edaravone (e.g. sold under the trade names Radicava® and Radicut®), dextromethorphan, anticholinergic medications, and psychiatric medications (e.g. antidepressants, antipsychotics, anxiolytics/hypnotics, mood stabilizers, and stimulants).

Neudexta® is a combination of dextromethorphan and quinidine, and can be used for the treatment of pseudobulbar affect (inappropriate laughing or crying). Anticholinergic medications and antidepressants can be used for treating excessive salivation. Examplary anticholinergic medications include glycopyrrolate, scopolamine, atropine (Atropen), belladonna alkaloids, benztropine mesylate (Cogentin), clidinium, cyclopentol ate (Cyclogyl), darifenacin (Enablex), dicylomine, fesoterodine (Toviaz), tiavoxate (Urispas), glycopyrrolate, homatropine hydrobromide, hyoscyamine (Levsinex), ipratropium (Atrovent), orphena.drine, oxybutynin (Ditropan XL), propantheline (Pro-banthine), scopolamine, methscopolamine, solifenacin (VESicare), tiotropium (Spiriva), tolterodine (Detrol), ttihexyphenidyl, trospium and diphenhydramine (Benadryl). Exemplary antidepressants include selective serotonin inhibitors, serotonin-norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors, and NMDA receptor antagonists.

The additional therapeutic agent(s) can be administered for a period of time before administering the initial dose of a composition comprising a bile acid or a pharmaceutically acceptable salt thereof (e.g., TURSO) and a phenylbutyrate compound (e.g., sodium phenylbutyrate), and/or for a period of time after administering the final dose of the composition. In some embodiments, a subject in the methods described herein has been previously treated with one or more additional therapeutic agents (e.g., any of the additional therapeutic agents described herein, such as riluzole and edavarone). In some embodiments, the subject has been administered a stable dose of the therapeutic agents) riluzole and/or edaravone) for at least 30 days (e.g., at least 40 days, 50 days, 60 days, 90 days, or 120 days) prior to administering the composition of the present disclosure. The absorption, metabolism, and/or excretion of the additional therapeutic agent(s) may be affected by the bile acid or a pharmaceutically acceptable salt thereof and/or the phenylbutyrate compound. For instance, co-administration of sodium phenylbutyrate with riluzole, or edavarone, may increase the subject's exposure to riluzole or edavarone. Co-administering riluzole with the bile acid or a pharmaceutically acceptable salt thereof and the phenylbutyrate compound can improve riluzole tolerance by the subject as compared to administering riluzole alone.

The combination of a bile acid or a pharmaceutically acceptable salt thereof, a phenylbutyrate compound, and one or more additional therapeutic agents can have a synergistic effect in treating ALS. Smaller doses of the additional therapeutic agents may be required to obtain the same pharmacological effect, when administered in combination with a bile acid or a pharmaceutically acceptable salt thereof, and a phenylbutyrate compound. In some embodiments, the amount of the additional therapeutic agent(s) administered in combination with a bile acid or a pharmaceutically acceptable salt thereof and a phenylbutyrate compound can be reduced by at least about 10% (e.g., at least about 15%, 20%, 25%, 30%, 35%, 40%, 45%, 50%, or 55%) compared to the dosage amount used when the additional therapeutic agent(s) is administered alone. Additionally or alternatively, the methods of the present disclosure can reduce the required frequency of administration of other therapeutic agents (e.g., other ALS therapeutic agents) to obtain the same pharmacological effect.

The bile acid or a pharmaceutically acceptable salt thereof and the phenylbutyrate compound can be administered shortly after a meal (e.g., within two hours of a meal) or under fasting conditions. The subject may have consumed food items (e.g., solid foods or liquid foods) less than 2 hours before administration of a bile acid or a pharmaceutically acceptable salt thereof and/or a phenylbutyrate compound; or will consume food items less than 2 hours after administration of one or both of the compounds. Food items may affect the rate and extent of absorption of the bile acid or a pharmaceutically acceptable salt thereof and/or the phenylbutyrate compound. For instance, food can change the bioavailability of the compounds by delaying gastric emptying, stimulating bile flow, changing gastrointestinal pH, increasing splanchnic blood flow, changing luminal metabolism of the substance, or physically or chemically interacting with a dosage form or the substance. The nutrient and caloric contents of the meal, the meal volume, and the meal temperature can cause physiological changes in the GI tract in a way that affects drug transit time, luminal dissolution, drug permeability, and systemic availability. In general, meals that are high in total calories and fat content are more likely to affect the GI physiology and thereby result in a larger effect on the bioavailability of a drug. The methods provided herein can further include administering to the subject a plurality of food items, for example, less than 2 hours less than 1.5 hour, 1 hour, or 0.5 hour) before or after administering the bile acid or a pharmaceutically acceptable salt thereof, and/or the phenylbutyrate compound.

### EXAMPLES

Additional embodiments are disclosed in further detail in the following examples, which are provided by way of illustration and are not in any way intended to limit the scope of this disclosure or the claims.

### Example 1

### Evaluation of the Safety, Tolerability, Efficacy and Activity of AMX0035, a Fixed Combination of Phenylbutyrate (PB) and Tauroursodeoxycholic Acid (TUDCA), for Treatment of ALS

### 1. Summary

### 1.1 Study Objectives and Endpoints

This study was intended as a proof of concept of AMX0035 as a safe and effective treatment of adult subjects with ALS. The main strategic objectives of this study are below.

The primary outcome measures are:
1. To confirm the safety and tolerability of a fixed-dose combination of PB and TUDCA in subjects with ALS over a 6-month period;
2. To measure the impact of the treatment using the slope of progression with the revised Amyotrophic Lateral Sclerosis Functional Rating Scale (ALSFRS-R);
   The secondary objectives of the study are:
1. To assess the impact of AMX0035 on the rate of decline of isometric muscle strength, as measured by the Accurate Test of Limb Isometric Strength (ATLIS);
2. To assess the impact of AMX0035 on disease progression as measured by Slow Vital Capacity (SVC) decline, time to tracheostomy and survival;
3. To assess the impact of AMX0035 on biomarkers including phosphorylated axonal neurofilament H subunit (pNT-H) levels and 18 kDa translocator protein (TSPO) uptake;
4. To develop concentration-response models of TUDCA and phenylbutyrate at steady-state after administration of AINIX0035 sachet twice-daily.
5. To measure the impact of AMX0035 on survival.

### 1.2 Study Design

This was a multicenter, randomized, double-blind, placebo-controlled 28-week study evaluating the safety, tolerability, efficacy, pharmacokinetics and biological activity of AVX0035.

### 1.3 Study Population

This study was conducted in subjects who have sporadic or familial ALS diagnosed as definite as defined by revised El Escorial criteria (Example 3). Subjects must provide written informed consent prior to screening. At screening, eligible subjects must be at least 18 years old and less than 80 years old, and have a VC≥60% of predicted capacity for age, height and gender. Subjects must have had onset of ALS symptoms less than or equal to 18 months prior to the screening visit, defined as first onset of weakness. Subjects on a stable dose of riluzole and those not taking riluzole, and women of child-bearing age at screening are eligible for inclusion as long as they meet specific protocol requirements. There will be no restrictions for subjects taking Radicava (edaravone) at the time of screening, or if started while enrolled in the study. Detailed criteria are described in the body of the protocol.

### 2. Study Outcome Measures

### 2.1 Primary Outcome Measures

The primary outcome measures for the study included:
Safety and tolerability defined as the proportion of subjects able to remain on study drug until planned discontinuation.
The rate of decline (slope of decline) in the ALS functional rating scale (ALSFRS-R).

Safety and tolerability were assessed by the procedures outlined in Section 7. The revised version of the ALSFRS was created to add assessments of respiratory dysfunction, including dyspnea, orthopnea, and the need for ventilatory support. The revised ALSFRS (ALSFRS-R) has been demonstrated to retain the properties of the original scale and show strong internal consistency and construct validity. Survival endpoint was defined as death, tracheostomy or permanent assisted ventilation (>22 hours a day).

### 2.2 Secondary Outcome Measures

Assessing the impact of MX0035 on the rate of decline of isometric muscle strength, as measured by the Accurate Test of Limb Isometric Strength (ATLIS);
Assessing the impact of AMX0035 on disease progression as measured by Slow Vital Capacity (SVC) decline;
Assessing the impact of AMX0035 on survival, hospitalization and tracheostomies;
Assessing the impact of AMX0035 on biomarkers including phosphotylated axonal neurofilament H subunit (pNF-H) levels and 18 kDa translocator protein (TSPO) uptake; and
Assessing the concentration-response model of TUDCA and phenylbutyrate at steady-state after administration of AMX0035 4 grams twice daily.

### 3. Study Design

### 3.1 A Overall Study Design and Plan

During the enrollment period approximately 176 subjects were screened from approximately 25 Northeast ALS Consortium (NEALS) centers in the US. One hundred thirty-seven (137) of these subjects were randomly assigned in a 2:1 ratio to oral (or feeding tube) twice daily sachet of active therapy or matching placebo. Treatment duration was twenty-four (24) weeks. For the first three weeks study drug was administered once daily. If tolerated, the dose was then increased to twice a day. Clinic visits occurred at Screening, Baseline, Week 3 (day 21), Week 6 (day 42), Week 12 (day 84), Week 18 (Day 126), and. Week 24 (Day 168). Phone calls were conducted at Week 9, Week 15, Week 21 and Week 28 (4 weeks after completion of treatment).

All visit windows were consecutive calendar days and were calculated from the day the subject started study treatment (Day 0, the day of the Baseline Visit). Any change from this visit window was considered an out of window visit deviation. A one thirty-two (132) week Open Label Extension (OLE) study was available to those subjects who completed the randomized, double-blind study (See Example 2).

### 3.2 Study Duration

Subjects remained on randomized, placebo-controlled, double-blind treatment until the Week 24 visit. Each randomized subject also had a follow-up telephone interview 28 days after the completion of dosing to assess for adverse events (AEs), changes in concomitant medications and to administer the ALSFRS-R. Including the Screening and Follow-up Visits, each subject was in the study for approximately 8 months.

### 4. Study Enrollment and Withdrawal

4A Inclusion and Exclusion criteria

### 4.1.1 Inclusion Criteria

1. Male or female, aged 18-80 years of age
2. Sporadic or familial ALS diagnosed as definite as defined by the World Federation of Neurology revised El Escorial criteria
3. Less than or equal to 18 months since ALS symptom onset
4. Capable of providing informed consent and following trial procedures
5. Geographically accessible to the site
6. Slow Vital Capacity (SVC)>60% of predicted value for gender, height, and age at the Screening Visit
7. Subjects must either not take riluzole or be on a stable dose of riluzole for at least days prior to the Screening Visit. Riluzole-naive subjects are permitted in the study.
8. Women of child bearing potential (e.g., not post-menopausal for at least one year or surgically sterile) must agree to use adequate birth control for the duration of the study and 3 months after last dose of study drug
   a. Women must not be planning to become pregnant for the duration of the study and 3 months after last dose of study drug
9. Men must agree to practice contraception for the duration of the study and 3 months after last dose of study drug
   a. Men must not plan to father a child or provide sperm for donation for the duration of the study and 3 months after last dose of study drug

Acceptable birth control methods for use in this study are:
Hortnonal methods, such as birth control pills, patches, injections, vaginal ring, or implants
Barrier methods (such as a condom or diaphragm) used with a spermicide (a foam, cream, or gel that kills sperm)
Intrauterine device (IUD)
Abstinence (no heterosexual sex)
Unique partner who is surgically sterile (men) or not of child bearing potential (female)

### Date of ALS Symptom Onset

For the purposes of this study, the date of symptom onset was defined as the date the subject first had symptoms of their disease, i.e., weakness. To be eligible for this study, the date of symptom onset must be no greater than exactly 18 months prior to the Screening Visit date.

### MR-PET Sub-Study

A subset of study subjects underwent MR-PET and were required to meet the following additional inclusion criteria:
1. Ability to safely lie flat for 90 min for MR-PET procedures in the opinion of the Site Investigator
2. High or mixed affinity to bind TSPS) protein (Genotype Ala/Ala or Ala/Thr)

### TSPO Affinity Test

Venous blood for the TSPO affinity test was drawn from all subjects who have indicated their interest in participating in the MR-PET sub-study (via a checkbox on the consent form). The blood was drawn at Screening in order to have the subjects genotyped for the Ala147Thr TSPO polymorphism in the TSPO gene (rs6971). About 10% of humans show low binding affinity to PBR28 (Zurcher et al. Increased in vivo glial activation in subjects with amyotrophic lateral sclerosis: Assessed with [¹¹C]-PBR28. Neuroimage Clin. 2015). High or Mixed affinity binders (Ala/Ala or Ala/Thr) were considered eligible, whereas low affinity binders (Thr/Thr) were considered ineligible for the MR-PET sub-study. A subject may be eligible for the main study but ineligible for the MR-PET sub-study. However, if a subject was found to be ineligible for the main study, he or she was automatically ineligible for the MR-PET sub-study as well.

### 4.1.2 Exclusion Criteria

Study subjects meeting any of the following criteria during screening evaluations were excluded from entry into the study:
1. Presence of tracheostomy
2. Exposure to PB, TUDCA or UDCA within 3 months prior to the Screening Visit or planning to use these medications during the course of the study
3. History of known allergy to PB or bile salts
4. Abnormal liver function defined as AST and/or ALT>3 times the upper limit of the normal
5. Renal insufficiency as defined by eGFR<60 mL/min/1.73 m².
6. Poorly controlled arterial hypertension (SBP>160 mmHg or DBP>100 mmHg) at the Screening Visit
7. Pregnant women or women currently breastfeeding
8. History of cholecystectomy
9. Biliary disease which impedes biliary flow including active cholecystitis, primary biliary cirrhosis, sclerosing cholangitis, gallbladder cancer, gallbladder polyps, gangrene of the gallbladder, abscess of the gallbladder.
10. History of Class III/IV heart failure (per New York Heart Association NYHA)
11. Severe pancreatic or intestinal disorders that may alter the enterohepatic circulation and absorption of TUDCA including biliary infections, pancreatitis and ileal resection
12. The presence of unstable psychiatric disease, cognitive impairment, dementia or substance abuse that would impair ability of the subject to provide informed consent, according to Site Investigator judgment
13. Patients who have cancer with the exception of the following: basal cell carcinoma. or successfully treated squamous cell carcinoma of the skin; cervical carcinoma in situ; prostatic carcinoma in situ; or other malignancies curatively treated and with no evidence of disease recurrence for at least 3 years
14. Clinically significant unstable medical condition (other than ALS) that would pose a risk to the subject if they were to participate in the study
15. Active participation in an ALS clinical trial evaluating an experimental small molecule within 30 days of the Screening Visit. (Please refer to MOP section E. Protocol Compliance for current list of experimental small molecules).
16. Exposure at any time to any cell therapies and gene therapies under investigation for the treatment of subjects with ALS (off-label use or investigational)
17. Exposure to monoclonal antibodies under investigation for the treatment of ALS (off-label use or investigational) within 90 days from screening. If previously exposed to monoclonal antibodies under investigation for the treatment of ALS, a 90-day wash-out period will be required prior to screening.
18. Implantation of Diaphragm Pacing System (DPS)
19. Anything that, in the opinion of the Site Investigator, would place the subject at increased risk or preclude the subject's full compliance with or completion of the study
20. Exposure to any disallowed medications listed below

### MR-PET Sub-Study

A subset of study subjects underwent MR-PET. The following additional exclusion criteria apply to this subset:
1. Exposure to immunomodulatory medications within 30 days of the Screening Visit
2. Any contraindication to undergo MRI studies such as:
   a. History of a cardiac pacemaker or pacemaker wires
   b. Metallic particles in the body
   c. Vascular clips in the head
   d. Prosthetic heart valves
   e. Severe claustrophobia impeding ability to participate in an imaging study
3. Low affinity binders (Thr/Thr) on the TSPO Affinity Test
4. Radiation exposure that exceeds the site's current guidelines

A subject may be eligible for the main study but ineligible for the MR-PET sub-study. However, if a subject was found to be ineligible for the main study, he or she was automatically ineligible for the MR-PET sub-study as well.

Benzodiazepines for MR-PET Sub-Study Subjects: If an MR-PET subject is taking a benzodiazepine, he or she should not take the benzodiazepine for at least 1 day before his or her scans with the exception of lorazepam and clonazepam that do not need to be discontinued.

Disallowed medications for all subjects include
HDAC Inhibitors including:
   Valproate
   Vorinostat (Zolinza)
   Romidepsin
   Chidamide
   Panobinostat
   Lithium
   Butyrate
   Suramin
Probenecid
Bile Acid Sequestrants including:
   Cholestyramine and Cholestyramine Light
   Questran and Questran Light
   Welchol
   Colestid and Colestid Flavored
   Prevalite

### Antacids Within Two Hours of AMX0035 Administration

Antacids containing Aluminum hydroxide or smectite (aluminum oxide) may not be taken within two hours of administration of AMX0035 as they inhibit absorption of TUDCA. These include:
Alamag
Alumina and Magnesia
Antacid, Antacid M and Antacid Suspension
Gen-Alox
Kudrox
Maalox HRF and Maalox TC
Magnalox
Madroxal
Mylanta and Mylanta. Ultimate
Ri-Mox
Rulox

### Mexiletine

Subjects who participated in the Mexiletine trial within the last 30 days were excluded from the trial. However, if a subject was using Mexiletine at a dosage less than or equal to 300 mg/day for cramps and fasciculations, the subject would not be excluded.

There is potential for an interaction between AMX0035 and Mexiletine. At 20 times the intended clinical concentration (Cₘₐₓ), the principal metabolite of Phenylbutyrate, Phenylacetylacetate has been shown to be inhibitory to CYP 1A2 and CYP 2D6 which are the major enzymes responsible for the breakdown of Mexiletine. Therefore, it is possible the co-administration of Phenylbutyrate and Mexiletine will increase the subject's exposure to Mexiletine.

Subjects who are co-administered AMX0035 and Mexiletine should therefore be monitored for Mexiletine-associated adverse events, and if these events present, the Site Investigator should consider stopping or reducing the dosage of Mexiletine. Adverse events associated with Mexiletine include but are not limited to cardiac arrhythmias, liver injury, and. blood dyscrasias.

### 4.3 Treatment Assignment Procedure

each subject who met all eligibility criteria was randomized to receive either therapy by twice daily sachet of AMX0035 (3 g PB and 1 g TUDCA) or matching placebo for 24 weeks of treatment. For the first three weeks of the study, subjects only took a single sachet daily and were instructed to increase to 2 sachets daily at the Week 3 Visit.

### 4.4 Reasons for Withdrawal

Any clinical adverse event (AE), laboratory abnormality, requirement for a concomitant medication, concurrent illness, or other medical condition or situation occurs such that, in the opinion of the investigator, continued participation in the study would not be in the best interest of the subject.
The subject is non-compliant or is lost-to-follow-up.

### 5. Treatments Administered

### 5.1.1 Study Product Description

AMX0035 is a combination therapy comprised of two active pharmaceutical ingredients, sodium phenylbutyrate (PB and tauroursodeoxycholic acid (TUDCA). Phenylbutyrate is an approved compound in the United States for urea cycle disorders and is marketed in the US as Buphenyl®. There is an existing USP monograph for this material. The drug substance PB is produced by Sri Krishna Pharmaceuticals, Ltd. under cGMP conditions. The manufacture and controls for PBA are described in Drug Master File No. 019569. The specifications for PB are identical to those of the Ph.Eur.

The chemical structure for PB is provided below.

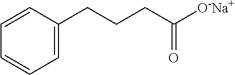

The drug substance TUDCA is currently marketed under the brand name Tudcabil and Taurolite. It is used for the indications of treatment of cholesterol gallstones. It has been used for the treatment of cholestatic liver diseases including primary cirrhosis, pediatric familial intrahepatic cholestasis, primary sclerosing cholangitis, and cholestasis due to cystic fibrosis.

The chemical structure for TUDCA is provided below.

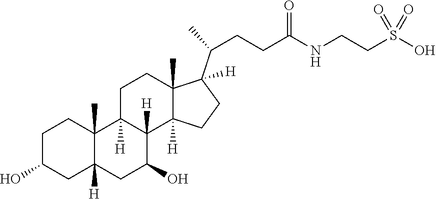

The drug substance TUDCA is produced by Prodotti Chimici F. Alimentaria S.p.A. The specifications for TUDCA are identical to those used by the supplier.

A powder filled sachet was used as the ANIX0035 drug product The drug product was filled under cGMP conditions in an aluminum foil lined sachet

The sachet containing active ingredients included:
a Active Ingredients:
   1 g TUDCA
   3 g PB
Excipients
   Sodium Phosphate Dibasic, Anhydrous
   Dextrates, Hydrates
   Sorbitol
   Syloid 63FP (colloidal silica)
   Sucralose
   Sodium Stearyl Furnarate
   Weber Mixed Berry Flavoring
   Kleptose Linecaps (maltodextrin)

### 5.1.2 Placebo

A matched placebo was used to maintain the dosage-blind. The placebo sachets for this study matched the corresponding AMX0035 sachets in size, color, and presentation. Administration of matching placebo was the same as for subjects in the treatment group. The placebo sachets contained:
Excipients
   Sodium Phosphate Dibasic, Anhydrous
   Dextrates, Hydrates
   Sorbitol
   Syloid 63FP (colloidal silica)
   Sucralose
   Sodium Stearyl Fumarate
   Weber Mixed Berry Flavoring
   Kleptose Linecaps (maltodextrin)
   Denatonium Benzoate Granules

### 5.2 Product Storage and Stability

All investigational drug supplies were kept at ambient temperature 15-25° C. Subjects were asked to store the kits containing the sachets away from moisture at room temperature. Stability has been assessed both at ICH standard and accelerated conditions for each of the individual active ingredients and they were found to be stable over five years. Drug product received regular stability testing over the course of the study to ensure product did not degrade.

### 5.3 Dosage, Preparation and Administration of Study Intervention/Investigational Product

It was recommended that the study drug be taken prior to a meal. Subjects should rip open the sachet of study drug and add it to a cup or other container and add approximately 8 oz. (1 cup) of room temperature water and stir vigorously. The study drug mixture should be consumed completely and within one hour of combining the contents of the sachet with water. Subjects may resume normal eating and drinking after taking the study drug.

### 5.3.1 Feeding Tube Study Drug Administration

For subjects with a gastrostomy or nasogastric (feeding) tube, the study drug may be dissolved in water as per the procedures outlined above in Section 5.3 and the study drug may be administered via the feeding tube.

### 5.4 Prior and Concomitant Therapy

Any investigational small molecule therapy being used or evaluated for the treatment of ALS is prohibited beginning 30 days prior to the Screening Visit and throughout, the study: This includes, but is not limited to, the following:
Fioglitazone
Arimoclomol
Olanzapine
Tamoxifen
NP001
Mexiletine
Rasagiline

Masitinib
Dexpramipexole
Tirasemtiv
Ibudilast
TW001
Inosine
RNS60
Acetyl-L-Carnitine
Methylcobalamine (if administered at doses equal to or greater than 25 mg per week)

Use of any biologic therapy prior to this study excludes subjects from enrollment. This includes any cell or gene therapy under evaluation for the treatment of ALS and includes but is not limited to, the following:
ISIS 333611
Zonis SOD1R
NurOwn
QCells
NSI-566
GM604
GSK 1223249
Treg cell therapies

### 5.4.1 Prohibited Medications and Contraindications

Agents which might impair bile acid processing or renal function are contraindicated with AMX0035. Prohibited medications include but are not limited to:
HDAC Inhibitors including:
   Valproate
   Vorinostat (Zolinza)
   Romidepsin
   Chidamide
   Panobinostat
   Lithium
   Butyrate
   Suramin
Probenecid for potential kidney interaction
Antacids containing aluminum hydroxide or smectite (aluminum oxide) within two hours of administration of AMX0035. These inhibit absorption of TUDCA. These include:
   Alamag
   Alumina and Magnesia
   Antacid, Antacid M and Antacid Suspension
   Gen-Alox
   Kudrox
   M.A.H.
   Maalox HRF and Maalox TC
   Magnalox
   Madroxal
   Mylanta and Mylanta Ultimate
   Ri-Mox
   Rulox
Bile Acid Sequestrants including:
   Cholestyramine and Cholestyramine Light
   Questran and Questran Light
   Welchol
   Colestid and Colestid Flavored
   Prevalite

### 6. Clinical Assessments and Outcome Measures

### 6.1 Clinical Variables

Assessments were performed at designated time-points throughout the study for clinical evaluation. In addition to the assessments evaluated below, subjects provided information on their demographics, past medical history, including ALS and cardiac history, as well as concomitant medication usage.

### 6.1:1 Vital Signs, Height & Weight

Vital signs were obtained after the subject had been in a seated position for several minutes. Vital signs, including systolic and diastolic blood pressure, pulse rate (radial artery)/minute, respiratory rate/minute, temperature and weight were assessed at specified visits. Height was measured and recorded at the Screening Visit only.

### 6.1.2 Clinical Laboratory Assessments

Hematology with differential panel: complete blood count with differential (hematocrit, hemoglobin, platelet count, RBC indices, Total RBC, Total WBC, and WBC & differential)
Blood chemistry panel/Liver function tests (LFTs): alanine aminotransferase (ALT (SGPT)), aspartate aminotransferase (AST (SGOT)), albumin, alkaline phosphatase, bicarbonate, blood urea nitrogen, calcium, chloride, creatinine, glucose, magnesium, phosphate, potassium, sodium, total bilirubin and total protein
Urinalysis: albumin, bilirubin, blood, clarity, color, glucose, ketones, nitrate, pH, protein, specific gravity, urobilinogen and WBC screen
Serum human chorionic gonadotrophin (hCG) for women of childbearing potential (WOCBP) (collected only at Screening Visit, and as necessary throughout course of study)

### 6.1.3 Biomarkers and Pharmacokinetic Analysis

Subjects had blood drawn to assess AMX0035 concentrations for pharmacokinetics (PK) pre-dose at the Baseline Visit and then again at either 1 hour or 4 hours (±10 minutes) post-dose at the Week 12 and 24 visits.

Additionally, blood was collected for biomarker analysis, including light and heavy neurofilament testing (NF-L and pNF-H, respectively). Neurofilaments was used as a mechanistic measure of neuronal death. NT-L and pNF-H were tested over multiple time points to generate a longitudinal dataset correlating neurofilament levels to observed clinical outcomes.

### 6.1.4 12-Lead Electrocardiogram (ECG)

A standard 12-lead ECG was performed and recorded.

### 6.1.5 Physical Examination

A comprehensive physical examination was performed and recorded.

### 6.1.6 Neurological Examination

A neurological examination was performed and recorded. Examination included assessment of mental status, cranial nerves, motor and sensory function, reflexes, coordination, and stance/gait.

### 6.1.7 Upper Motor Neuron-Burden (UMN-B)

The Penn Upper Motor Neuron-Burden (UMN-B) is the total number of pathological LMN signs on examination including pathologically brisk biceps, supinator, triceps, finger, knee and ankle reflexes, and extensor plantar responses assessed bilaterally and brisk facial and jaw jerks. The scale is a combination of Ashworth, Reflexes, and Pseudobulbar Affect scale (Range score: 0-32). The UMN also includes scoring of the Center for Neurologic Study-Lability Scale (CNS-LS), a 7-item self-report scale that assesses pseudobulbar affect (PBA) by measuring the perceived frequency of PBA episodes (laughing or crying). Data was generated from the clinical exam and scored from 1-5, the lowest score indicating normal tone and the highest extreme spasticity.

### 6.1.8 Columbia Suicide Severity Rating Scale (C-SSRS)

The C-SSRS involves a series of probing questions to inquire about possible suicidal thinking and behavior. At the Baseline Visit, the C-SSRS Baseline version was administered. This version is used to assess suicidality over the subject's lifetime: At all clinic visits after the Baseline Visit, the Since Last Visit version of the C-SSRS was administered. This version of the scale assesses suicidality since the subject's last visit.

### 6.1.9 Adverse Events

Adverse events (AEs), if any, were documented at each study visit, including the Screening Visit once the informed consent form has been signed by the subject, and at all study visits, including the Final Telephone Call 28 days (+5 days) after the last dose of study drug. Information on adverse effects of study drug and on inter-current events was determined at each visit by direct questioning of the subjects, review of concomitant medications, and vital sign results.

### 6.2 Outcome Measures

### 6.2.1 ALSFRS-R (Amyotrophic Lateral Sclerosis Functional Rating Scale—Revised)

The ALSFRS-R is a quickly administered (5 minutes) ordinal rating scale (ratings 0-4) used to determine subjects' assessment of their capability and independence in 12 functional activities. All 12 activities are relevant in ALS. Initial validity was established by documenting that in ALS subjects, change in ALSFRS-R scores correlated with change in strength over time, was closely associated with quality of life measures, and predicted survival. The test-retest reliability is greater than 0.88 for all test items. The advantages of the ALSFRS-R are that the categories are relevant to ALS, it is a sensitive and reliable tool for assessing activities of daily living function in those with ALS, and it is quickly administered. With appropriate training the ALSFRS-R can be administered with high inter-rater reliability and test-retest reliability. The ALSFRS-R can be administered by phone with good inter-rater and test-retest reliability. The equivalency of phone versus in-person testing, and the equivalency of study subject versus caregiver responses have also recently been established. The ALSFRS-R therefore may also be given to the study subject over the phone.

### 6.2.2 Pulmonary Function Testing Slow Vital Capacity (CVC)

The vital capacity (VC) (percent of predicted normal) was determined using the upright slow VC method. The VC can be measured using conventional spirometers that have had a calibration check prior to subject testing. Three VC trials were required for each testing session, however up to 5 trials may be performed if the variability between the highest and second highest VC is 10% or greater for the first 3 trials. Only the 3 best trials were recorded on the CRF. The highest VC recorded was utilized for eligibility,

### 6.2.3 Isometric Strength Testing (Accurate Testing of Limb Isometric Strength, or ATLIS)

Isometric strength was measured using the Accurate Testing of Limb isometric Strength device (ATLIS) developed by Dr. Patricia Andres of Massachusetts General Hospital. The device was specifically designed to alleviate the reproducibility concerns that exist for prior strength measurements such as hand held dynamometry (HHD). ATLIS does not depend on experimenter strength, and has measurement settings to ensure that subjects are in the same position each time they are tested. ATLIS may detect functional decline before the ALSFRS-R, which may have a ceiling effect, and may be able to detect changes in function with greater sensitivity to ALSFRS-R. The measure does show a small training effect, so measurement at initial screening visit was included to allow subjects to become acquainted with the device.

### 6.2.4 Neuroimaging MR-PET Sub-Study

A subset of subjects underwent MR-PET scans at the Baseline Visit and again between the Week 12 and 21 Visits. Prior to the scan, every MR-PET sub-study subject completed the MR-PET Safety Questionnaire.

### 6.2.5 Survival Assessment

Survival endpoint was considered as mortality, tracheostomy or permanent assisted ventilation.

### 7. Safety and Adverse Events

The adverse event (AE) definitions and reporting procedures provided in this protocol comply with all applicable United States Food and Drug Administration (FDA) regulations and International Conference on Harmonization (ICH) guidelines. The Site Investigator will carefully monitor each subject throughout the study for possible adverse events. All Abs will be documented on CRFs designed specifically for this purpose. It is also important to report all AEs, especially those that result in permanent discontinuation of the investigational product being studied, whether serious or non-serious.

### 7.1 Definitions of AES, Suspected Adverse Drug Reactions & SAES

### 7.1.1 Adverse Event and Suspected Adverse Drug Reactions

An adverse event (AE) is any unfavorable and unintended sign (including a clinically significant abnormal laboratory finding, for example), symptom, or disease temporally associated with a study, use of a drug product or device whether or not considered related to the drug product or device.

Adverse drug reactions (ADR) are all noxious and unintended responses to a medicinal product related to any dose. The phrase “responses to a medicinal product” means that a causal relationship between a medicinal product and an adverse event is at least a reasonable possibility, i.e., the relationship cannot be ruled out. Therefore, a subset of AEs can be classified as suspected ADRs, if there is a causal relationship to the medicinal product.

Examples of adverse events include: new conditions, worsening of pre-existing conditions, linically significant abnormal physical examination signs (i.e. skin rash, peripheral edema, etc), or clinically significant abnormal test results (i.e. lab values or vital signs), with the exception of outcome measure results, which are not being recorded as adverse events in this trial (they are being collected, but analyzed separately). Stable chronic conditions (i.e., diabetes, arthritis) that are present prior to the start of the study and do not worsen during the trial are NOT considered adverse events. Chronic conditions that occur more frequently (for intermittent conditions) or with greater severity, would be considered as worsened and therefore would be recorded as adverse events.

Adverse events are generally detected in two ways:
Clinical→symptoms reported by the subject or signs detected on examination.
Ancillary Tests→abnormalities of vital signs, laboratory tests, and other diagnostic procedures (other than the outcome measures, the results of which are not being captured as AEs).
   For the purposes of this study, symptoms of progression:/worsening of ALS, including ‘normal’ progression, will be recorded as adverse events. The following measures of disease progression will not be recorded as adverse events even if they worsen (they are being recorded and analyzed separately): vital capacity results, ALSFRS-R, and ATLIS results.

If discernible at the time of completing the AL log, a specific disease or syndrome rather than individual associated signs and symptoms should be identified by the Site Investigator and recorded on the AE log. However, if an observed or reported sign, symptom, or clinically significant laboratory anomaly is not considered by the Site Investigator to be a component of a specific disease or syndrome, then it should be recorded as a separate AE on the AE log. Clinically significant laboratory abnormalities, such as those that require intervention, are those that are identified as such by the Site Investigator.

Subjects will be monitored for adverse events from the time they sign consent until completion of their participation in the study (defined as death, consent withdrawal, loss to follow up, early study termination for other reasons or following completion of the entire study).

An unexpected adverse event is any adverse event, the specificity or severity of which is not consistent with the current Investigator's Brochure. An unexpected, suspected adverse drug reaction is any unexpected adverse event for which, in the opinion of the Site Investigator or Sponsor (or their designee), there is a reasonable possibility that the investigational product caused the event.

### 7.1.2 Serious Adverse Events

A serious adverse event (SAE) is defined as an adverse event that meets any of the following criteria:
1. Results in death.
2. Is life threatening: that is, poses an immediate risk of death as the event occurred.
   a. This serious criterion applies if the study subject, in the view of the Site investigator or Sponsor, is at immediate risk of death from the AL as it occurs. It does not apply if an AE hypothetically might have caused death if it were more severe.
3. Requires in-patient hospitalization or prolongation of existing hospitalization.
   a. Hospitalization for an elective procedure (including elective PEG tube/g-tube/feeding tube placement) or a routinely scheduled treatment is not an SAE by this criterion because an elective or scheduled “procedure” or a “treatment” is not an untoward medical occurrence.
4. Results in persistent or significant disability or incapacity.
   a. This serious criterion applies if the “disability” caused by the reported AL results in a substantial disruption of the subject's ability to carry out normal life functions.
5. Results in congenital anomaly or birth defect in the offspring of the subject (whether the subject is male or female).
6. Necessitates medical or surgical intervention to preclude permanent impairment of a body function or permanent damage to a body structure.
7. Important medical events that may not result in death, are not life-threatening, or do not require hospitalization may also be considered SAEs when, based upon appropriate medical judgment, they may jeopardize the subject and may require medical or surgical intervention to prevent one of the outcomes listed in this definition. Examples of such medical events include blood dyscrasias or convulsions that do not result in in-patient hospitalization, or the development of drug dependency or drug abuse.

An in-patient hospital admission in the absence of a precipitating, treatment-emergent, clinical adverse event may meet criteria for “seriousness” but is not an adverse experience, and will therefore, not be considered an SAE. An example of this would include a social admission (subject admitted for other reasons than medical, e.g., lives far from the hospital, has no place to sleep).

A serious, suspected adverse drug reaction (SUSAR) is an SAE for which, in the opinion of the Site investigator or Sponsor, there is a reasonable possibility that the investigational product caused the event. The Site Investigator is responsible for classifying adverse events as serious or non-serious.

### 7.3 Adverse Events and Serious Adverse Events—Reportable Events

The following are considered reportable events and must be reported to the Medical Monitor and Coordination Center within 24 hours of the site being notified of the event.
All events that meet the above criteria for Serious Adverse Events (SAES)
Dosage Changes (Dose Management)
   Investigational Product Suspension, Reduction or Re-challenge
   a Investigational Product Discontinuation
Key Study Events:
   a Subject Final Disposition
   Feeding Tube Placement
   Permanent Assisted Ventilation (PAV)*
   Tracheostomy
   Mortality
   Pregnancy
   Diaphragm Pacing System (DPS) device implantation
   Emergency or Accidental Unblinding Events
Permanent Assisted Ventilation (PANT) is defined as more than 22 hours daily of non-invasive mechanical ventilation for more than one week (7 days). The date of onset of PAV is the first day of the seven days.

### 8. Statistical Considerations

### 8.1 Statistical Methods

Analysis of the PROACT and ceftriaxone de-identified subject databases suggests that statistical powering can be significantly improved by enrolling subjects who are <1.5 years from symptom onset and have a definite diagnosis of ALS according to El Escorial Criteria. Mixed-effects modeling was used to account for both the variance between subjects and the deviation within subjects from their average rate of decline.

Power for safety and tolerability was considered in three ways: incidence of adverse events (AEs), change in ALFSR-R and ATLIS, and change in biomarker such as pNF-H. With 88 treated subjects, we will have an 80% probability of detecting any adverse event expected to occur in at least 2% of treated subjects. We will have 80% power to detect a 28 percentage point elevation in the rate of any adverse event relative to placebo based on a one-tailed test at alpha=0.05. We will consider a dose tolerable if the proportion of treatment failures (discontinuation of study drug due to an adverse event) is less than 40% with 80% confidence, one-tailed. With 88 treated subjects this would occur if 30 or fewer subjects on AMX0035 fail to complete the 6-month study. By this criterion, we will have 80% power for declaring AMX0035 tolerable at the tested dose if the true treatment failure rate is 30%.

A shared-baseline, mixed-effects analysis was used for primary analysis. A covariate of bulbar onset or onset elsewhere and a second covariate of age at enrollment was included in the analysis. The mixed-effects model accounts for both the variance between subjects and the deviation within subjects from their average rate of decline. The same analysis was used for clinical outcomes in this trial. An alpha of 0.05 was used for testing.

### 8.2 Analysis for Safety

The safety data was summarized by treatment group. Treatment AEs was coded and graded using MedDRA grading criteria. The treatment groups were compared with respect to occurrence of each adverse event and incidence of Grade III/IV adverse events. Total number of serious adverse events and abnormal laboratory tests were compared between groups using Fisher's exact test. Withdrawal, abnormal laboratofy tests, vital signs and use of concomitant medications were assessed to characterize the safety profile of the combination of PB and TUDCA. Compliance data were determined for each visit and by treatment group. The time to subject refusal were compared between treatment groups to better determine tolerability. This was accomplished using a method of survival analysis that allows informative censoring due to death. Descriptive statistics denoting the changes from baseline to the final assessment visit with respect to key laboratory parameters and vital signs was also provided.

### 8.3 Analysis for Efficacy

Modified intention-to-treat analysis was performed, including all randomized subjects receiving at least one dose of the study medication and having at least one primary efficacy assessment after randomization. Slope was imputed from available data and time points. Homogeneity of clinical characteristics and efficacy variables at baseline between the two randomization groups (between-group baseline differences) were assessed by analysis of variance for continuous variables and by a chi-squared test for discrete variables. All efficacy endpoints were compared between the two randomization groups at study end (between-group differences at study end) by means of analysis of covariance for continuous variables, adjusting for baseline value and for center effect, and by a chi-squared test for discrete variables. Survival time was compared between treatments by a Kaplan-Meier survival analysis.

The primary analysis strategy used a shared-baseline, mixed-effects model of ALSFRS-R progression rate. The mixed-effects model accounts for both the valiance between subjects and the deviation within subjects from their average rate of decline. The same analysis was used for clinical outcomes in this trial. An alpha of 0.05 was used for testing. An effect size (slowing of ALSFRS-R slope) greater than 30% was tested.

### 8.4 Analysis Populations

The modified intent to treat (ITT) population included all study subjects who are randomized and receive at least one dose of study drug. The ITT population was considered for primary analyses. For ITT analyses, subjects were grouped based on randomized treatment, regardless of treatment actually received.

### Example 2

### Open Label Extension Study

To determine the long-term safety of AMX0035 in subjects with ALS, an open label extension study is carried out.

### Study Design and Plan

This is a multicenter, open label extension, up to 132-week study evaluating the long-term safety of AMX0035. Up to 132 subjects that participated in the randomized, double-blind trial will be able to enroll in this study. Subjects will be given oral (or feeding tube) twice daily sachet of active therapy. Treatment duration will be up to one thirty-two (132) weeks starting at the Screening/Ba.seline visit. Clinic visits will occur at Screening/Baseline, Week 6 (day 42), Week 12 (day 84), Week 24 (day 168), Week 36 (day 252), and Week 52 (Day 364), Week 68 (Day 476), Week 84 (Day 588), Week 100 (Day 700), Week 116 (Day 812), Week 132 (Day 924).

All visit windows are consecutive calendar days and are calculated from the day the subject starts study treatment (Day 0, the day of the Screening/Baseline Visit). The screening/Baseline visit must occur within 28 days of the Week 24 visit of the main study. If the Screening/Baseline visit occurs on the day of the Week 24 visit or within 7 days of that visit then it is not necessary to complete the assessments, labs and outcomes. If the Screening/Baseline visit occurs Day 8-Day 28 then all assessments, labs and outcomes need to be completed. Visit windows will be +/−10 days for the Week 6 and Week 12 visits and +/−28 days for the Week 24, Week 36, Week 52, Week 68, Week 84, Week 100, Week 116 and Week 132 visits. Any change from this visit window will be considered an out of window visit deviation.

### Study Objectives

The primary objective of the study is to assess long-term safety of oral (or feeding tube) administration of AMX0035 via sachet (3 g PB and 1 g TUDCA) twice daily for compassionate use.

The primary outcome measure is:
1. To confirm the long-term safety of AMX0035 in subjects with ALS over a 132-week period
   Secondary outcome measures will include:
1. The rate of key study events including tracheostomy, hospitalization, and death
2. Rate of progression on the ALSFRS-R scale
3. ATLIS rate of progression
4. late of progression of slow vital capacity

### Study Population

This study will be conducted in subjects who have sporadic or familial ALS diagnosed as definite as defined by revised El Escorial criteria (See Example 3). Subjects must provide written informed consent prior to screening. At screening/baseline subjects must have completed participation in the randomized, double-blind trial.

### Study Enrollment

### Inclusion Criteria:

1. Completion of all visits in the randomized, double blind. AMX0035 study. Subjects that receive tracheostomy or PAV during the course of the main study will still be followed as ITT until the week 24 visit before enrollment in the OLE.
2. Must enroll in the OLE within 28 days of the Week 24 visit of the main study.
3. Signed informed consent to enter the open label extension phase. Exclusion Criteria:
1. Discontinued study drug prematurely in the double-blind phase of the study for reasons other than tracheostomy or PAV.
2. Exposure to or anticipated requirement for any disallowed medication listed below.
3. Any ongoing adverse events that in the opinion of the Site Investigator are clear contraindications to the study drug.
4. Unstable cardiac or other life-threatening disease emergent during the randomized, double blind study.
5. Any major medical condition that in the opinion of the Site Investigator would interfere with the study and place the subject at increased risk.

Subjects who receive tracheostomy or PAV while in the randomized, double-blind trial can elect to enroll in the OLE so long as they complete all visits in the main study.

Disallowed medications for all subjects include:
HDAC Inhibitors including:
   Valproate
   Vorinostat (Zolinza)
   Romidepsin
   Chidamide
   Panobinostat
   Lithium
   Butyrate
   Suramin
Probenecid
Bile Acid. Sequestrants including:
   Cholestyramine and Cholestyramine Light
   Questran and Questran Light
   Weichol
   Colestid and Colestid Flavored
   Prevalite

### Antacids Within Two Hours of Study Drug Administration

Antacids containing Aluminum hydroxide or smectite (aluminum oxide) may not be taken within two hours of administration of the study drug as they inhibit absorption of TETDCA. These include: Alamag, Alumina and Magnesia, Antacid, Antacid M and Antacid Suspension, Gen-Alox, Kudrox, M. A. H., Maalox FIRE and Maalox TC, Magnalox, Madroxal, Mylanta and Mylanta Ultimate. Ri-Mux, and Rulox.

### Study Drug and Treatment Administration

A new formulation is used for the open label extension which has been optimized for better taste. A powder filled sachet is used as the AMX0035 drug product, and the drug product is filled under cGMP conditions in an aluminum foil lined sachet. The sachet containing active ingredients include:
Active Ingredients:
   1 g TUDCA
   3 g PB (Phenybutyrate)
Excipients
   Dextrates
   Sorbitol
   Sucralose
   Syloid 63FP (colloidal silica)
   Kleptose Linecaps (maltodextrin)
   Firmenich Flavor Masking Flavorairt
   Firmenich Mixed Berry Flavorant
   Sodium Phosphate Dibasic
   Sodium Stearyl Fumarate

Changes from the batch used in the randomized, double blind study include a different level of sucralose, the mixed berry flavor being provided by a new company and the addition of a flavor masking agent. Study drug will be provided in clinic on the day of the screening/baseline visit and re-supplied at each subsequent visit. Subjects will take 2 sachets daily, 1 sachet in the morning and 1 sachet in the afternoon, throughout the study.

### Duration of Treatment and Follow-Up

Subjects will remain on treatment until the Week 132 or early discontinuation visit.

### Study Schedule

### Screening/Baseline Clinic Visit:

Day 0 Visit of the open label extension sub-study may be the same as Week 24 Visit of the main study—so that exams and tests do not need to be duplicated if they were previously completed.

The following procedures will be performed:
Obtain written informed consent from subject
Assess inclusion and exclusion criteria
Review and document concomitant medications and therapies
Administer C-SSRS (Baseline Version)
Administer ALSFRS-R questionnaire
Perform pulmonary function testing including slow vital capacity (SVC)* Note height should be recorded from the main study Screening Visit.
Measure isometric strength using ATLIS machine
Assess and document adverse events (AEs) that occur after subject signs informed consent form (ICF)
Measure vital signs (blood pressure, heart and breathing rates, temperature, and weight)
Perform 12-lead ECG (Electrocardiogram)
[After other tests] Collect blood samples for clinical laboratory assessments including Hematology (CBC with differential), Complete Chemistry Panel, Liver Function Tests, serum pregnancy test (for women of child-bearing potential [WOCBP]), optional DNA analysis if not completed during main study
Collect urine sample for urinalysis
Dispense 2 kits of study drug (12 weeks +2 weeks extra)
Capture key study events
Schedule the Week 6 Visit

### Week 6, Week 12, Week 24, Week 36, Week 52, Week 68, Week 84, Week 100, Week 116, Week 132 or Early Discontinuation/Final Safely Clinic Visit:

The Week 6 and. Week 12 visits will take place +1- 10 days and the Week 24, Week 36, Week 52, Week 68, Week 84, Week 100, Week 116 and Week 132 visits will take place +/−28 days from the time specified in the schedule of activities (table as beginning of this section).

The following procedures will be performed:
Review and assess Adverse Events
Measure vital signs
Administer the C-SSRS questionnaire (Since Last Visit)
Administer ALSFRS-R questionnaire
Perform pulmonary function testing including slow vital capacity (SVC)
Measure isometric strength using KILTS machine
Perform 12-lead ECG (Electrocardiogram)
a Collect blood samples for clinical laboratory assessments including Hematology (CBC with differential), Complete Chemistry Panel, Liver Function Tests, optional DNA analysis if not completed during main study
Collect urine sample for urinalysis
Perform study drug accountability
Dispense study drug (Except at Week 132/Early Discontinuation)
Capture key study events
Schedule next study visit (Except Week 132/Early Discontinuation)

### Laboratory Testing

The following laboratory tests will be performed for safety:
Hematology with differential panel: complete blood count with differential (hematocrit, hemoglobin, platelet count, RBC indices, Total RBC, Total WBC, and WBC & differential)
Blood chemistry panel/Liver function tests (LFTs): alanine aminotransferase (ALT (SGPT)), aspartate aminotransferase (AST (SGOT)), albumin, alkaline phosphatase, bicarbonate, blood urea nitrogen, calcium, chloride, creatinine, glucose, potassium, sodium, total bilirubin and total protein
Urinalysis:, bilirubin, blood, clarity, color, glucose, ketones, nitrate, pH, protein, specific gravity, urobilinogen and WBC screen
Serum human chorionic gonadotrophin (hCG) for women of childbearing potential (WOCBP) (collected only at Screening Visit, and as necessary throughout course of study)

### Example 3

### El Escorial World Federation of Neurology Criteria for the Diagnosis of ALS

Information Obtained from the web site: www,wfnals.org: The diagnosis of Amyotrophic Lateral Sclerosis [ALS] requires:

A—The presence of:
P (A:1) evidence of lower motor neuron (LMN) degeneration by clinical, electrophysiology or neuropathologic examination,
(A:2) evidence of upper motor neuron (UMN) degeneration by clinical examination, and
(A:3) progressive spread of symptoms or signs within a region or to other regions, as determined by history or examination, together with
   B—The absence of:
(B:1) electrophysiological and pathological evidence of other disease processes that might explain the signs of LIEN and/or UMN degeneration, and
(B:2) neuroimaging evidence of other disease processes that might explain the observed clinical and electrophysiological signs.

### Clinical Studies in the Diagnosis of ALS

A careful history, physical and neurological examination must search for clinical evidence of UMN and LMN signs in four regions [brainstem, cervical, thoracic, or lumbosacral spinal cord] (see Table 1) of the central nervous system [CNS]. Ancillary tests should be reasonably applied, as clinically indicated, to exclude other disease processes. These should include electrodiagnostic, neurophysiological, neuroimaging and clinical laboratory studies. Clinical evidence of LMN and UMN degeneration is required for the diagnosis of ALS. The clinical diagnosis of ALS, without pathological confirmation, may be categorized into various levels of certainty by clinical assessment alone depending on the presence of UMN and LMN signs together in the same topographical anatomic region in either the brainstem [bulbar cranial motor neurons], cervical, thoracic, or lumbosacral spinal cord [anterior horn motor neurons]. The terms Clinical Definite ALS and Clinically Probable ALS are used to describe these categories of clinical diagnostic certainty on clinical criteria alone:
A. Clinically Definite ALS is defined on clinical evidence alone by the presence of UMN, as well as LMN signs, in three regions.
B. Clinically Probable ALS is defined on clinical evidence alone by UMN and LMN signs in at least two regions with some LMN signs necessarily rostral to (above) the LMN signs.
C. Clinically Probable ALS—Laboratory-supported is defined when clinical signs of UMN and LMN dysfunction are in only one region, or when LMN signs alone are present in one region, and LMN signs defined by EMU criteria are present in at least two limbs, with proper application of neuroimaging and clinical laboratory protocols to exclude other causes.
D. Clinically Possible ALS is defined when clinical signs of LMN and LMN dysfunction are found together in only one region or UMN signs are found alone in two or more regions; or LMN signs are found rostral to LMN signs and the diagnosis of Clinically Probable—Laboratory-supported ALS cannot be proven by evidence on clinical grounds in conjunction with electrodiagnostic, neurophysiologic, neuroimaging or clinical laboratory studies. Other diagnoses must have been excluded to accept a diagnosis of Clinically Possible ALS.

TABLE 1

| | Brainstem | Cervical | Thoracic | Lumbosacral |
|---|---|---|---|---|
| Lower motor | jaw, face, | neck, arm, | back, | back, abdomen, |
| neuron signs | palate, | hand, | abdomen | leg, foot |
| weakness, | tongue, larynx | diaphragm | | |
| atrophy, | | | | |
| fasciculations | | | | |
| Upper motor | clonic jaw | clonic DTRs | loss of | clonic DTRs - |
| neuron signs | gag reflex | Hoffman reflex | superficial | extensor plantar |
| pathologic spread | exaggerated | pathologic | abdominal | response |
| of reflexes, clonus, | snout reflex | DTRs | reflexes | pathologic DTRs |
| etc. | pseudobulbar | spastic tone | pathologic | spastic tone |
| | features | preserved reflex | DTRs | preserved reflex in |
| | forced yawning | in weak wasted | spastic tone | weak wasted limb |
| | pathologic | limb | | |
| | DTRs | | | |
| | spastic tone | | | |

### Example 4

### ALS Functional Rating Scale-Revised (ALSFRS-R)

### Example 5

### Analysis of Trial Results

### Trial Parficipants

To increase statistical power to detect differences in the rate of decline in the Amyotrophic Lateral Sclerosis Functional Rating Scale Revised (ALSFRS-R), we defined inclusion criteria to enroll individuals with ALS who were within 18 months from symptom onset and had a diagnosis of definite ALS as described by revised El Escorial criteria (i.e., clinical evidence of both upper and lower motor neuron signs in at least three body regions) (See, e.g., Brooks et al. Amyotroph Lateral Scler Other Motor Neuron Disord 2000; 1:293-9). These criteria were chosen to select for a population of participants with fast-progressing ALS, based on an analysis in historical cohorts from previously conducted clinical investigations (Section 2.1). Such selection has two potential benefits: one, reducing the heterogeneity of the rate of disease progression among participants, thereby increasing statistical power, and two, selecting for a population with faster-than-average disease progression, allowing for a more rapid analysis of efficacy.

Additional eligibility criteria included age 18 to 80 years; slow vital capacity (SVC) exceeding 60% of the predicted value for an individual's age, sex, and height; and either no use of riluzole at trial entry or a stable dosage of riluzole for at least 30 days prior to screening. After edaravone became available in August 2017, the protocol was amended to allow for use of edaravone prior to and during the trial.

Exclusion criteria included the presence of a tracheostomy or diaphragm pacing system, history of active participation in an ALS clinical trial evaluating an experimental small molecule within 30 days of screening, and any of the following exposures: sodium phenylbutyrate, taurursodiol, or ursodiol within 3 months prior to screening (or previously planned use of any of these individual agents during the course of the trial); any investigational cell or gene therapies at any time; or monoclonal antibodies within 90 days before screening.

### Trial Interventions and Procedures

Eligible participants were randomized in a 2:1 ratio to receive either sodium phenylbutyrate/taurursodiol (ANIX0035; 3 g sodium phenylbutyrate and 1 g taurursodiol per sachet) or matching placebo, administered orally or by feeding tube once daily, for a planned duration of 24 weeks. The two-drug co-formulation and placebo were provided in single-use sachets as a powder to be dissolved in room-temperature water before administering. The powders were constituted to look, dissolve, and taste the same. Participants were instructed to take one sachet per day for the first 3 weeks and two sachets per day (one in morning and one in evening) thereafter, if tolerated. Clinic or phone visits were conducted at baseline and every 3 weeks thereafter through week 24, with a final phone follow-up at week 28. Participants who completed the randomized, double-blind trial were eligible for enrollment in an open-label extension trial for up to 132 weeks evaluating the long-term safety of sodium phenylbutyra.teltaurursodiol (NCT03488524).

### Outcomes

The primary efficacy outcome was the rate (slope) of decline in the ALSFRS-R total score from baseline through trial end at week 24. The ALSFRS-R consists of 12 items across four subdomains of bodily function (bulbar, fine motor, gross motor, and breathing), with each item being scored on an ordinal scale (0=total loss of function, 4=no loss of function, maximum 48, lower scores indicating greater difficulty with function) (See, e.g., Cedarbaum et al, J Neurol Sci 1999; 169:13-21). The scale is validated for administration in person or by telephone and has shown high inter- and intrarater reliability. The rates of decline in ALSFRS-R subdomain scores were evaluated as exploratory efficacy outcomes. Secondary clinical efficacy outcomes (in hierarchical order) included the rate of decline in isometric muscle strength as measured by the Accurate Test of Limb Isometric Strength (AIL′S) device; rate of decline in SVC; and rates of death or death-equivalent events (tracheostomy or permanent assisted ventilation [>22 hours daily for >7 days]), tracheostomy only, and hospitalization (except for elective surgeries) over the 24-week treatment duration (See Paganoni et al. Clin lrrvesti (Load) 2014; 4:605-18). A pharmacokinetic analysis was also included as a prespecified secondary outcome. Change in blood levels of phosphorylated neurofilament heavy chain protein, abiomarker of motor neuron degeneration, from baseline to week 24 was assessed as a secondary biological outcome (See Poesen et al. Front Neurol 2019; 9:1167).

Isometric muscle strength of six upper and six lower extremity muscle groups was assessed using the ATLIS device, with three trials of each muscle group. Raw values were standardized to percentage of predicted normal (PPN) strength based on age, sex, weight, and height (See, e.g. Andres et al. Muscle Nerve 2013; 47:177-82). Standardized PPN scores for the highest recorded force for each muscle group were averaged to yield total, upper, and lower summary scores. (Further details regarding ATLIS are provided in Section 2.4 below.) Respiratory muscle function was assessed by SVC, measured in an upright position for at least three trials per assessment or for up to five trials when the highest and second highest of the first three measurements differed by 10% or more. SVC volumes were standardized to PPN based on age, sex, and height. The highest recorded SVC score from all attempts was utilized for analysis.

Safety was assessed via documentation of treatment-emergent adverse events (TEAEs) at each trial visit. Symptoms of ALS progression, including those consistent with disease progression, were recorded as TEAEs. Any worsening of a measure of disease progression that was being recorded and analyzed separately (i.e., ALSFRS-R, ATLIS, and SVC) was not recorded as a TEAE Trial drug was considered tolerable if the proportion of participants discontinuing the drug due to TEAEs was less than 40% with 80% confidence, one-tailed.

Trial drug adherence was assessed by having participants return their empty and unused sachets at each clinic visit. Adherence was defined as taking more than 80% or less than 125% of anticipated trial drug as determined by sachet counts.*Means±SD. †Adherence is calculated as the number of empty sachets returned/total number of sachets (empty ±unused).

An exit questionnaire was administered at the final trial visit (week 24 or at early discontinuation) to evaluate blinding of participants and investigators to treatment allocation by asking whether they thought the participant was on active treatment or placebo.

### Statistical Analysis

To calculate sample size, an analysis was conducted on the first 6 months of data from participants in a large historical trial (the ceftriaxone trial) who met the previously described fast-progressing criteria. Use of a shared-baseline, mixed-effects regression model was assumed, with no added model covariates. This analysis found that, with a 2:1 randomization ratio between treatment and placebo, approximately 131 participants followed over 6 months would provide 80% power to detect a 30% treatment effect on the ALSFRS-R total score when tested at a two-sided alpha of 0.1. It was expected that including terms in the model for pre-baseline ALSFRS-R slope and age, as covariates for the slope over time, and adding increased assessment frequency (nine assessments over 6 months in CENTAUR vs. four assessments over 6 months in the ceftriaxone trial) would add additional power, allowing for the use of the prespecified two-sided alpha level of 0.05.

Safety analyses were performed in the safety population, consisting of all participants who received at least one dose of trial drug. The primary population for efficacy analyses was the modified intent-to-treat (mITT) population, consisting of all participants who received at least one dose of trial drug and had at least one ALSFRS-R total score recorded after randomization. A post hoc analysis of the intent-to-treat (ITT) population, including two participants in the active group who did not undergo a post-baseline efficacy assessment and were excluded from the mITT population, was also performed. Additional pre-specified efficacy analyses were performed in the on-drug population, consisting of all participants in the mITT population but excluding data from any trial visits that occurred more than 30 days after trial drug termination or temporary interruption and excluding one participant whose administration of any trial drug could not be confirmed.

A hierarchy was prepared for secondary outcomes for inference testing. ATLIS was the first secondary outcome in this hierarchy and included three separate measurements (upper extremity, lower extremity, and total scores) with no hierarchy specified for the separate ATLIS measurements. Because of this lack of hierarchy, our post hoc decision was to report unadjusted 95% confidence intervals for the three ATLIS measurements.

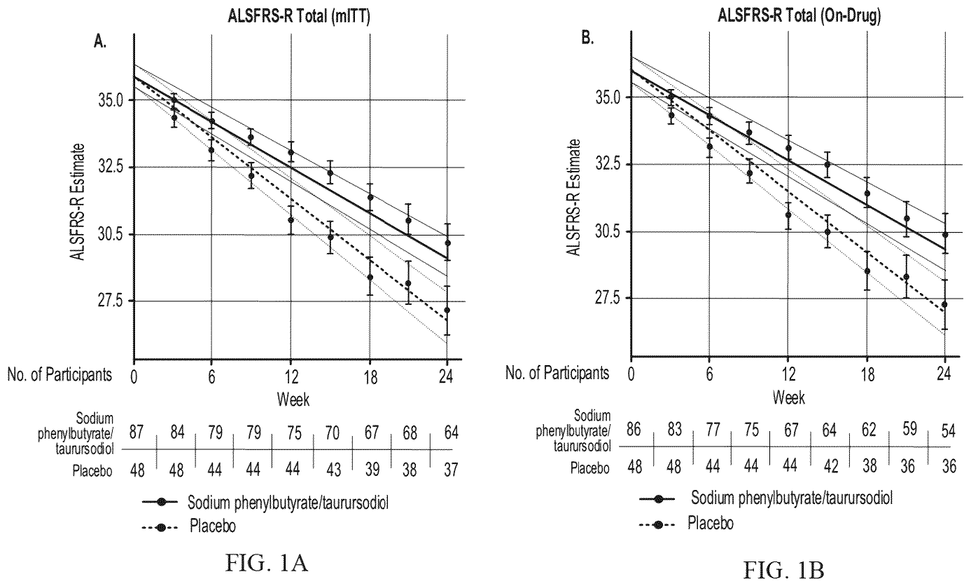

The absolute scores for all continuous efficacy outcomes were analyzed using a random-slope, shared-baseline, linear mixed model adjusted for age and pre-baseline ALSFRS-R slope (rate of decline in ALSFRS-R total score from ALS symptom onset to baseline), both covariates that have been shown to be relevant in historical data (See e.g., Labra et al. J Neurol Neurosurg Psychiatry 2016; 87:628-32; Daghlas et al. Amyotroph Lateral Scler Frontotemporal Degener 2018; 19:206-11; Taylor et al. Ann Clin Transl Neurol 2016; 3:866-75). Interaction terms between time and age and time and pre-baseline ALSFRS-R slope were included, reflecting our interest in slope differences. Analyses to confirm the linear model are described in Section 2.5 below. A post hoc mixed model that replaced continuous time with categorical visit was performed to generate separate estimates at each time point for purposes of visualizing visit-by-visit data over time (FIGS. 1A and 1B). These estimates assumed the same mean level of baseline covariates across both treatment groups.

FIGS. 1A and 1B show estimated Rate of Decline in ALSFRS-R Total Score Over 24 Weeks (Primary Outcome). FIG. 1A shows the treatment-dependent rates of decline in ALSFRS-R total score estimated in the mITT population in the primary analysis (solid line=sodium phenylbutyrateltaurursodiol, dashed line=placebo; lines immediately above and below each one reflect plus and minus one standard error). Overlaid on the estimated slopes from the primary analysis are visit-specific estimates (and standard error bars) from a post hoc shared-baseline, repeated-measures mixed model with the same adjustments but categorical time and unstructured covariance among repeated measures. FIG. 1B shows estimates from the same pair of models applied to the on-drug population. In the primary model, the mean slopes of the ALSFRS-R total score were sodium phenylbutyra.teltaurursodiol were −1.24 points/month vs. −1.66 points/month for active drug and placebo, respectively (difference=0.42 points/month; 95% CI, 0.03 to 0.81; P=0.03). Results were similar in the on-drug analysis, with mean slopes of ALSFRS-R total score of −1.22 points/month vs. -1.68 points/month for trial drug and placebo, respectively (difference=0.46 points/month; 95% Cl, 0.05 to 0.87; P=0.03), ALSFRS-R denotes Amyotrophic Lateral Sclerosis Functional Rating Scale Revised, ANOVA analysis of variance, mITT modified intent-to-treat.

Primary efficacy analyses used all available baseline and post-baseline data for all participants in the mITT sample, including those who discontinued trial drug but continued in the trial. For these analyses, no imputation was performed for missing data. Additional details regarding handling of missing data are provided in Section 2.5 below. In addition to the aforementioned post hoc ITT analysis, prespecified sensitivity analyses were performed to evaluate the effects of all missing data, data missing specifically due to death or death-equivalent events, and concomitant use of riluzole, edaravone, or both on the primary analysis (Section 2.5 below). A post hoc joint rank analysis was performed in the safety population to incorporate all survival events into the analysis of function (ALSFRS-R), providing adjusted estimates that accounted for potential bias due to death.

Analyses were performed using SAS (version 9.4, SAS Institute, Cary, NC). Tests were declared significant for two-tailed P≤0.05. The proportions of estimated assigned treatment (active, placebo, or missing) by participants and investigators, per their exit questionnaire responses, were compared within each treatment group using a chi-square statistic. The primary reasons for their estimates were also summarized by proportion.

### Example 6

### YKL-40 and CRP Biomarkers Endpoint

As shown above, the safety and efficacy of an oral, fixed-dose combination of sodium phenylbutyrate and taurursodiol (PB and TURSO) in ALS were evaluated in the multicenter phase 2 trial encompassing a randomised, placebo-controlled phase and an open-label extension long-term follow-up phase. The primary end point of the randomized phase of the trial was met, with PB and TURSO significantly slowing functional decline as measured by the Amyotrophic Lateral Sclerosis Functional Rating Scale-Revised (ALSFRS-R) over 24 weeks compared with placebo. To assess drug target engagement of PB and TUDCA, plasma levels of the YKL-40 biomarker and the CRP biomarker were analyzed to measure the effects of AMX0035 on pharmacodynamic and pathophysiological targets relevant to ALS. YKL-40 and CRP plasma levels were significantly reduced in participants with ALS receiving PB and TURSO, with reductions observed as early as week 12. YKL-40 concentration further correlated significantly with disease progression as measured by the ALSFRS-R, the most widely used functional end point in ALS trials and the primary efficacy outcome in the phase 2 trial.

Briefly, blood samples were drawn at baseline and every 6 weeks thereafter through week 24 (or early discontinuation) to obtain plasma. Immunoassays for YKL-40 and CRP were conducted in a blinded manner using 0.5-mL plasma samples. As for all prespecified efficacy outcomes in the Phase 2 trial, the participant population for these post hoc biomarker analyses was the modified intent-to-treat (mITT) population, consisting of all participants who received :1 dose of study medication and had >1 post-baseline ALSFRS-R assessment. Specifically, participants with >1 post-baseline plasma sample collected over the 24-week randomised phase duration were included in the analysis. Logto-transformed plasma biomarker measurements (Vu L, et al. J Neurol Neurosurg Psychiatry. 2020; 91(4):350-358) were analyzed using a random-slope, shared-baseline, and linear mixed effects model, with calculation of geometric least squares (LS) mean biomarker concentrations for each treatment group at pre-specified time points corresponding with sample collection. Change-from-baseline analyses that did not assume a shared baseline between treatment groups were also performed; the ratios of geometric LS mean biomarker concentrations relative to baseline concentration at each time point are presented below. For YKL-40, geometric mean ratios from a model without linear trend assumption were calculated for Weeks 12 and 24. Pearson correlation coefficients were calculated to assess the correlation between plasma YKL-40 concentration and both ALSFRS-R total score and ALSHRS-R slope.

YKL-40 is a secreted glycoprotein considered to be a biological marker of active gliosis and neuroinflammation. YKL-40 levels will therefore be assessed as a biomarker of neuroinflammation. YKL-40 is produced by astrocytes and is significantly elevated in subjects with MCI and mild Alzheimer's disease (Craig-Schapiro, Rebecca, et al, “YKL-40: a novel prognostic fluid biomarker for preclinical Alzheimer's disease.” Biological psychiatry 68.10 (2010): 903-912). YKL-40 also has evidence supporting its relevance in ALS. CSF samples from people with ALS have shown elevation in YKL4O compared to age matched controls (p=0.045). YKL-40 has been associated with inflammatory processes in neurodegenerative diseases (Llorens et al., 2017). The concentration of YKL-40 has been shown to correlate with ALS severity (Dreger, 2022; Oeckl, 2018; Thompson, 2019), speed of disease progression (Vu, 2000; Gille, 2019; Andres-Benito, 2018), and survival (Masrori, 2022). YKL-40 has also been shown to be correlated with ALSFRS-R progression as measured by ALSFRS-R slope (p<0.001) (Andres-Benito et al., 2018).

C-reactive protein (CRP) is an acute-phase protein regulated by proinflammatory cytokines and secreted by hepatocytes during the inflammatory response. C-reactive protein is referred to as a pentraxin because of its capacity to aggregate, in a noncovalent fashion, into flat pentameric discs. The pentraxins are presumed to have great survival value and to be intimately associated with innate immune defense. C-reactive protein is a biomarker of the inflammatory response with a significant prognostic value for several types of tumors, cardiovascular diseases, and rheumatic diseases. Studies have shown that CRP levels could be used as a prognostic measure for ALS patients (see, e.g., Lunetta C, Lizio A, Maestri E, Sansone V A, Mora G, Miller R G, Appel S H, Chiò A. Serum C-Reactive Protein as a Prognostic Biomarker in Amyotrophic Lateral Sclerosis. JAMA Neurol. 2017 Jun. 1; 74(0:660-667).

Of 135 total participants in the mITT population (PB and RASO, n=87; placebo, n=48), 126 (PB and TLTRSO, n=81; placebo, n=45) had plasma samples available for these analyses. A summary of the baseline plasma biomarker concentrations are shown in Table 2 below.

TABLE 2
Baseline plasma levels of YKL-40 and CRP biomarkers

| Biomarker, | | PB and TURSO | |
|---|---|---|---|
| (ng/mL) | Statistic | (AMX0035); (n = 81) | Placebo (n = 45) |
| YKL-40 | Mean (SD) | 43.9 (47.6) | 36.6 (24.1 |
| | Median | 29.5 | 28.2 |
| | Range | 10.0-268.6 | 11.4-126.1 |
| CRP | Mean (SD) | 3115.0 (3915.6) | 6460.5 (12,160.8) |
| | Median | 1526.1 | 2228.0 |
| | Range | 130.3-20,928.9 | 163.9-51,801.2 |

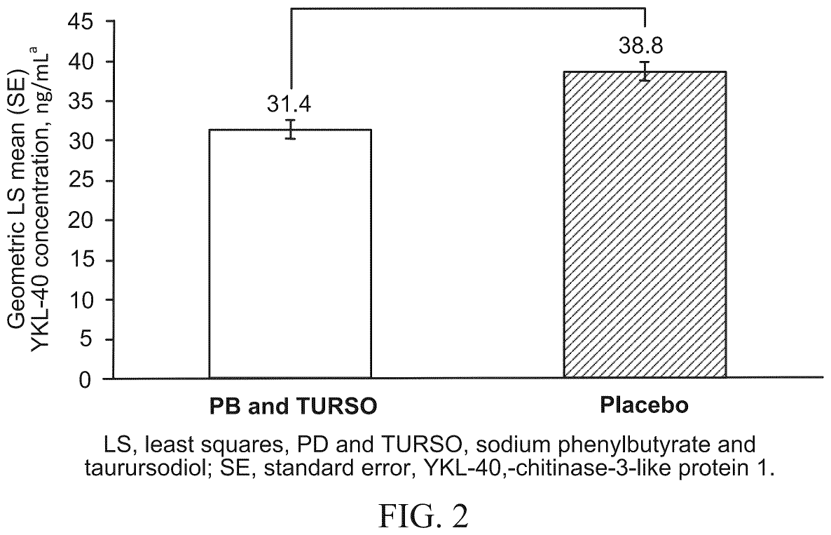

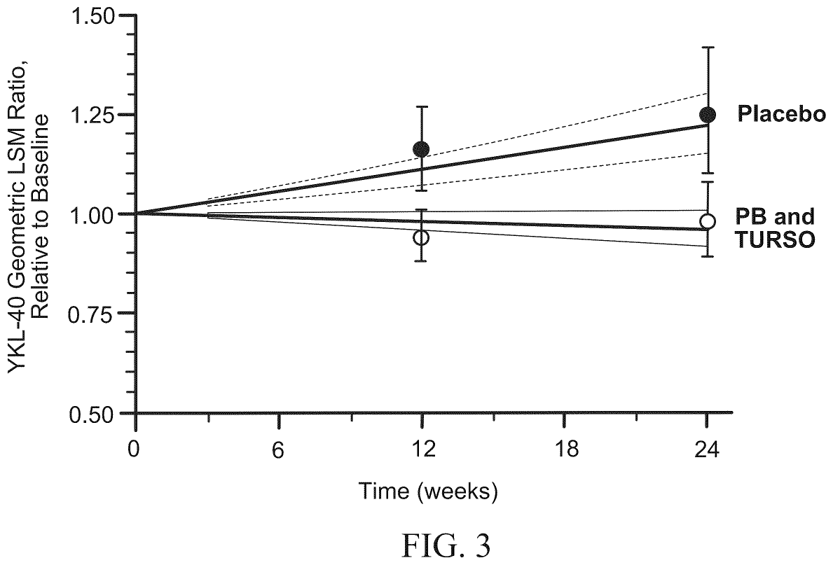

YKL-40 concentration correlated with both ALSFRS-R total score and Ail,,SFR.S-R slope. The correlation of YKL-40 to ALSFRS-R score was r=−0.21, p=0.0001 and the correlation of YKL-40 to ALSFRS-R progression rate was r=0.11, p=0.034. Additionally, Table 3 below shows that subjects treated with AMX0035 vs. those treated with the placebo had lower levels of YKL-40 (data also shown in the bar graph in FIG. 2). Geometric LS mean YKL-40 plasma concentration was 10% lower (ratio, 0.90; 95% CI, 0.83-0.97) at week 12 and approximately 20% lower (ratio, 0.81; 95% CI, 0.69-0.94) at week 24 in the PB and TURSO versus placebo group (P=0.008; Table 3). At the end of the 24-week randomized phase, a significant change from baseline was observed between the AMX0035 and placebo arms in plasma YKL-40 levels. The change-from-baseline analyses showed a significant reduction in plasma YKL-40 concentration in the AMX0035 versus placebo group (P=0.002; FIG. 3, which shows the geometric LS mean (SE) ratio relative to baseline). Specifically, in FIG. 3, Log10-transformed YKL-40 concentration measurements were analyzed using a random-slope, shared-baseline, linear mixed-effects model (colored lines, with shading reflecting SEs); geometric mean ratios (95% CIs) from a model without linear trend assumption are shown at weeks 12 and 24. The median change from baseline was −0.98 versus 4.83 for AMX0035 and placebo, respectively (P=023).

As shown in Table 3, geometric LS mean CRP concentration was 17% lower (ratio, 0.83; 95% CI, 0.69-1.00) at week 12 in the PB and TURSO group compared with the placebo group; geometric LS mean CRP concentration was approximately 30% lower in the PB and TURSO group (1833.6 ng/mL) compared with the placebo group (2650.2 ng/ml,) at week 24 (ratio, 0.69; 95% CI, 0.48-1.00; P=0.048). Furthermore, CRP concentration correlated with both ALSFRS-R total score (r of −0.19; P=0.0002) and ALSFRS-R slope (r of 0.21; P<0.0001). Change from baseline analyses showed a significant reduction in plasma CRP concentration in the PB and TURSO versus placebo (P=0.018).

TABLE 3
Plasma biomarker concentrations*

| | Baseline† | | Week 12‡ | | Week 24‡ | | |
|---|---|---|---|---|---|---|---|
| | PB and | | PB and | | PB and | | |
| | TURSO | Placebo | TURSO | Placebo | TURSO | Placebo | P |
| Biomarker | (n = 81) | (n = 45) | (n = 81) | (n = 45) | (n = 81) | (n = 45) | Value§ |
| YKL-40 | 43.9 | 36.6 | 31.6 | 35.2 | 31.4 | 38.8 | .008 |
| | (47.64) | (24.06) | (1.05) | (1.06) | (1.06) | (1.08) | |
| CRP | 3115.0 | 6460.5 | 1817.9 | 2185.6 | 1833.6 | 2650.2 | .048 |
| | (3915.59) | (12,160.81) | (1.11) | (1.12) | (1.14) | (1.18) | |

*All concentrations expressed in ng/mL.
†Mean (SD) concentration.
‡Geometric least-squares mean (SE) concentration (log10-transformed⁸) from a random-slope, shared-baseline, linear mixed-effects model.
§For comparison between PB and TURSO group versus placebo group, calculated using shared-baseline, mixed-effects model.
CRP, C-reactive protein; PB and TURSO, sodium phenylbutyrate and taurursodiol; SD, standard deviation; SE, standard error; YKL-40, chitinase-3-like protein 1.

1. A method of reducing the level of YKL-40 or C-reactive protein (CRP) in a biological sample of a human subject, the method comprising:
determining or having determined a level of YKL-40 or CRP in a biological sample of a human subject, and
administering to the human subject Taurursodiol (TURSO) and sodium phenylbutyrate.

2. The method of claim 1, wherein the human subject has one or more symptoms of ALS.

3. A method of treating at least one symptom of ALS in a human subject, the method comprising:
(a) determining or having determined a first level of YKL-40 in a first biological sample of a human subject,
(b) administering to the human subject TURSO and sodium phenylbutyrate,
(c) determining or having determined a second level of YKL-40 in a second biological sample of the human subject, and
(d) continuing administering to the human subject TURSO and sodium phenylbutyrate if the second level of YKL-40 is higher than the first level of YKL-40.

4. A method of treating at least one symptom of ALS in a human subject, the method comprising
(a) determining or having determined a first level of YKL-40 in a first biological sample of a human subject,
(b) administering to the human subject TURSO and sodium phenylbutyrate,
(c) determining or having determined a second level of YKL-40 in a second biological sample of the human subject, and
(d) continuing administering to the subject TURSO and sodium phenylbutyrate if the second level of YKL-40 is lower than the first level of YKL-40.

5. The method of claim 4, wherein step (b) comprises administering to the human subject TURSO and sodium phenylbutyrate for about 24 weeks and step (d) (2) comprises continuing administering to the human subject TURSO and sodium phenylbutyrate if the second level of YKL-40 is lower than the first level of YKL-40 by about 0.75 ng/mL or more.

6. The method of claim 4, wherein step (b) comprises administering to the human subject TURSO and sodium phenylbutyrate for about 24 weeks and step (d) (2) comprises continuing administering to the human subject TURSO and sodium phenylbutyrate if the second level of YKL-40 is lower than the first level of YKL-40 by about 0.75 ng/mL or less.

7. The method of claim 2, further comprising:
after administering to the human subject TURSO and sodium phenylbutyrate,
determining or having determined that a level of YKL-40 in a biological sample of the human subject is higher than about 33 ng/mL, and
continuing administering to the human subject TURSO and sodium phenylbutyrate.

8. A method of treating at least one symptom of ALS in a human subject, the method comprising:
(a) determining or having determined a first level of C-reactive protein (CRP) in a first biological sample of a human subject,
(b) administering to the human subject TURSO and sodium phenylbutyrate,
(c) determining or having determined a second level of CRP in a second biological sample of the human subject, and
(d) continuing administering to the subject TURSO and sodium phenylbutyrate if the second level of CRP is higher than the first level of CRP.

9. A method of treating at least one symptom of ALS in a human subject, the method comprising:
(a) determining or having determined a first level of C-reactive protein (CRP) in a first biological sample of a human subject,
(b) administering to the human subject TURSO and sodium phenylbutyrate,
(c) determining or having determined a second level of CRP in a second biological sample of the human subject, and
(d) continuing administering to the subject TURSO and sodium phenylbutyrate if the second level of CRP is lower than the first level of CRP.

10. The method of claim 2, further comprising:
after administering to the human subject TURSO and sodium phenylbutyrate,
determining or having determined that the level of C-reactive protein (CRP) in a biological sample of the subject is higher than about 1835 ng/mL, and
continuing administering to the subject TURSO and sodium phenylbutyrate.

11. The method of claim 4, wherein the TURSO is administered at a dose of about 10 mg/kg to about 50 mg/kg of the body weight of the subject.

12. The method of claim 4, wherein the sodium phenylbutyrate is administered at a dose of about 10 mg/kg to about 400 mg/kg of the body weight of the subject.

13. The method of claim 4, wherein the TURSO and the sodium phenylbutyrate are administered once a day or twice a day.

14. The method of claim 4, wherein the TURSO is administered at an amount of about 1 gram once a day or twice a day.

15. The method of claim 4, wherein the sodium phenylbutyrate is administered at an amount of about 3 grams once a day or twice a day.

16. The method of claim 4, wherein the TURSO and the sodium phenylbutyrate are administered separately.

17. The method of claim 4, wherein the TURSO and the sodium phenylbutyrate are administered concurrently.

18. The method of claim 4, wherein the TURSO and the sodium phenylbutyrate are administered orally or through a feeding tube.

19. The method of claim 4, wherein the TURSO is administered at an amount of about 1 gram once a day or twice a day orally or through a feeding tube, and the sodium phenylbutyrate is administered at an amount of about 3 grams once a day or twice a day orally or through a feeding tube.

20. The method of claim 4, wherein the method comprises administering about 1 gram of TURSO once a day and about 3 grams of sodium phenylbutyrate once a day for about 14 days, followed by administering about 1 gram of TURSO twice a day and about 3 grams of sodium phenylbutyrate twice a day.

21. The method of claim 4, wherein the TURSO and the sodium phenylbutyrate are formulated as a single powder formulation.

22. The method of claim 4, wherein the biological sample is a CSF sample or a serum sample.

23. The method of claim 4, wherein the human subject is diagnosed with ALS.

24. The method of claim 4, wherein the human subject is suspected as having ALS.
